(19) United States
(12) Patent Application Publication
(10) Pub. No.: US 2024/0382481 A1
(43) Pub. Date: Nov. 21, 2024

(54) SRPK INHIBITORS

(71) Applicant: Merck Patent GmbH, Darmstadt (DE)

(72) Inventors: Matthias Leiendecker, Darmstadt (DE); Timo Heinrich, Darmstadt (DE)

(73) Assignee: Merck Patent GmbH, Darmstadt (DE)

(21) Appl. No.: 18/291,529

(22) PCT Filed: Jul. 28, 2022
(86) PCT No.: PCT/EP2022/071160

§ 371 Date: Jan. 23, 2024

(30) Foreign Application Priority Data
Jul. 29, 2021 (EP) 21188490.3

(51) Int. Cl. A61K 31/506; A61K 9/08; A61K 31/444; A61K 31/519; C07D 401/04; C07D 401/14; C07D 403/14; C07D 471/04; C07D 487/04

(52) U.S. Cl. CPC A61K 31/506; A61K 9/08; A61K 31/444; A61K 31/519; C07D 401/04; C07D 401/14; C07D 403/14; C07D 471/04; C07D 487/04

(57) ABSTRACT

Compounds of formula (I)

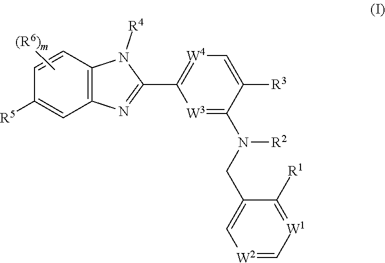

are made, wherein W¹, W², W³, W⁴, R¹, R², R³, R⁴, R⁵, R⁶ and m have the meaning as described herein. The compounds are SRPK inhibitors and can be used, inter alia, for the treatment of hyperproliferative disorders. A medicament and a pharmaceutical composition are made that include the compound of formula (I). Methods are developed for treating a disease caused, mediated, and/or propagated by SRPK activity, for preparing a medicament for the treatment thereof, and for inhibiting SRPK.

### CROSS-REFERENCE TO RELATED APPLICATIONS

This application is a National Stage entry under § 371 of International Application No. PCT/EP2022/071160, filed on Jul. 28, 2022, and which claims the benefit of priority to European Patent Application No. 21188490.3, filed on Jul. 29, 2021. The content of each of these applications is hereby incorporated by reference in its entirety.

### BACKGROUND OF THE INVENTION

### Field of the Invention

The present invention relates to compounds of formula (I)

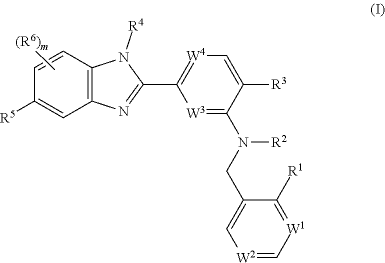

wherein W¹, W², W³, W⁴, R¹, R², R³, R⁴, R⁵, R⁶ and m have the meaning as described herein, and/or physiologically acceptable salts thereof. The compounds of formula (I) can be used as SRPK inhibitors. Objects of the invention are also pharmaceutical compositions comprising the compounds of formula (I), and the use of the compounds of formula (I) for the treatment of hyperproliferative disorders.

### Description of Related Art

Protein kinases constitute a large family of structurally related enzymes that are responsible for the control of a wide variety of signal transduction processes within the cell (G Hardie & S Hanks 1995, The Protein Kinase Facts Book. I and II, Academic Press, San Diego. CA). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks & Hunter, FASEB J 1995, 9: 576-596; Knighton et al., Science 1991, 253: 407-414; Hiles et al., Cell 1992, 70: 419-429; Kunz et al., Cell 1993, 73: 585-596; Garcia-Bustos et al., EMBO J 1994, 13: 2352-2361).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signaling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth, and differentiation factors, etc.), cell cycle events, environmental or nutritional stresses, etc. The appropriate protein kinases are involved in signaling pathways to activate or inactivate (either directly or indirectly), for example, a metabolic enzyme, regulatory protein, receptor, cytoskeletal protein, ion channel or pump, or transcription factor. Uncontrolled signaling due to defective control of protein phosphorylation has been implicated in several diseases, including, for example, inflammation, cancer, allergy/asthma, diseases and conditions of the immune system, diseases and conditions of the central nervous system, and angiogenesis.

Serine-arginine protein kinases (SRPKs) are a subfamily of serine-threonine kinases that phosphorylate serines in serine-arginine dipeptide motifs. SRPKs are described to alter constitutive and alternative mRNA splicing and maturation as well as chromatin reorganization in somatic and sperm cells, cell cycle and p53 regulation (T Giannakouros, E Nikolakaki, I Mylonis, E Georgatsou, FEBS Journal 2011, 278: 570-586). As SRPKs are associated with the promotion of cancer growth, SRPK inhibition could be an effective therapy (I P Nikas, S C Themistocleous, S A Paschou, K I Tsamis, H S Ryu, Cells 2020, 9(1): 19; G Wang, W Sheng, X Shi, X Li, J Zhou, M Dong, The FEBS Journal 2019, 286: 1668-1682; T Arends, J M Taliaferro, E Petermann, J R Knapp, B O'Connor, R M Torres, J R Hagman, bioRxiv 759829). The best characterized family members are SRPK1, SRPK2 and SRPK3.

Several different small molecules with SRPK inhibiting activity are described in the art. However, existing SRPK inhibitors lack potency and/or selectivity (T Fukuhara, T Hosoya, S Shimizu, K Sumi, T Oshiro, Y Yoshinaka, M Suzuki, N Yamamoto, L A Herzenberg, L A Herzenberg, M Hagiwara, PNAS 2006, 103(30): 11329-11333; R P Siqueira et Al., Eur J Med Chem 2017, 134: 97-109; J Batson et Al., ACS Chem. Biol. 2017, 12(3): 825-832; J M Hatcher, Cell Chem Bio 2018, 25(4): 460-470). Specifically, SRPK inhibitors typically show cross-reactivities against the structurally related CLK or DYRK proteins.

### SUMMARY OF THE INVENTION

The invention had the object of finding novel compounds having valuable properties, in particular those, which can be used for the preparation of medicaments. It has been surprisingly found that the compounds according to the invention and salts thereof have very valuable pharmacological properties while being well tolerated. In particular, they act as SRPK inhibitors. The invention relates to compounds of formula (I)

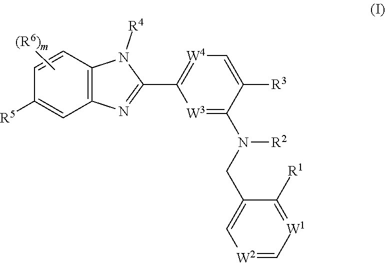

wherein
W¹, W², W³, W⁴ denote independently from one another N or CH;
R¹ denotes NYSO₂Y or Y;
R², R⁴ denote Y;
R³ denotes Y or Hal;
R², R³ together also denote —(CY)₂- or —(CR⁷)—(CY)₂;
R⁵, R⁶ denote independently from one another Hal, Y or Het;
R⁷ denotes Y or ═O;
Y denotes H or A;
A denotes unbranched or branched alkyl having 1-10 C atoms, in which 1-7 H atoms can be replaced independently from one another by Hal;
Het denotes an optionally substituted, saturated, unsaturated or aromatic monocyclic 5-6-membered heterocycle having 2-5 C atoms and 1-3 N, O and/or S atoms; Hal denotes F, Cl, Br or I; and
m denotes 0, 1, 2 or 3;
and/or a physiologically acceptable salt thereof;
with the proviso that

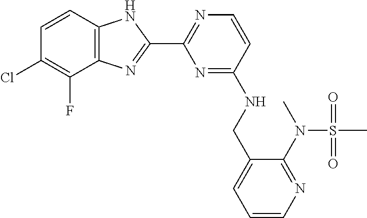 [SMILES: CN(c1ncccc1CNc1ccnc(-c2nc3c(F)c(Cl)ccc3[nH]2)n1)S(C)(=O)=O]

is excluded.

### DETAILED DESCRIPTION OF THE INVENTION

In the meaning of the present invention, the compound is defined to include pharmaceutically usable derivatives, solvates, prodrugs, tautomers, enantiomers, racemates and stereoisomers thereof, including mixtures thereof in all ratios.

The term “pharmaceutically usable derivatives” is taken to mean, for example, the salts of the compounds according to the invention and so-called prodrug compounds.

The term “solvates” of the compounds is taken to mean adductions of inert solvent molecules onto the compounds, which are formed owing to their mutual attractive force. Solvates are, for example, mono- or dihydrates or alkoxides. The invention also comprises solvates of salts of the compounds according to the invention.

The term “prodrug” is taken to mean compounds according to the invention which have been modified by means of, for example, alkyl or acyl groups, sugars or oligopeptides and which are rapidly cleaved in the organism to form the effective compounds according to the invention. These also include biodegradable polymer derivatives of the compounds according to the invention. It is likewise possible for the compounds of the invention to be in the form of any desired prodrugs such as, for example, esters, carbonates, carbamates, ureas, amides or phosphates, in which cases the actually biologically active form is released only through metabolism. Any compound that can be converted in-vivo to provide the bioactive agent (i.e., compounds of the invention) is a prodrug within the scope and spirit of the invention. Various forms of prodrugs are well known in the art and are described. It is further known that chemical substances are converted in the body into metabolites which may where appropriate likewise elicit the desired biological effect—in some circumstances even in more pronounced form. Any biologically active compound that was converted in-vivo by metabolism from any of the compounds of the invention is a metabolite within the scope and spirit of the invention.

The compounds of the invention may be present in the form of their double bond isomers as pure E or Z isomers, or in the form of mixtures of these double bond isomers. Where possible, the compounds of the invention may be in the form of the tautomers, such as keto-enol tautomers. All stereoisomers of the compounds of the invention are contemplated, either in a mixture or in pure or substantially pure form. The compounds of the invention can have asymmetric centers at any of the carbon atoms. Consequently, they can exist in the form of their racemates, in the form of the pure enantiomers and/or diastereomers or in the form of mixtures of these enantiomers and/or diastereomers. The mixtures may have any desired mixing ratio of the stereoisomers. Thus, for example, the compounds of the invention which have one or more centers of chirality and which occur as racemates or as diastereomer mixtures can be fractionated by methods known per se into their optical pure isomers, i.e. enantiomers or diastereomers. The separation of the compounds of the invention can take place by column separation on chiral or nonchiral phases or by recrystallization from an optionally optically active solvent or with use of an optically active acid or base or by derivatization with an optically active reagent such as, for example, an optically active alcohol, and subsequent elimination of the radical.

The invention also relates to the use of mixtures of the compounds according to the invention, for example mixtures of two diastereomers, for example in the ratio 1:1, 1:2, 1:3, 1:4, 1:5, 1:10, 1:100 or 1:1000. These are particularly preferably mixtures of stereoisomeric compounds.

The nomenclature as used herein for defining compounds, especially the compounds according to the invention, is in general based on the rules of the IUPAC-organization for chemical compounds and especially organic compounds. The terms indicated for explanation of the above compounds of the invention always, unless indicated otherwise in the description, have the following meanings:

The term “unsubstituted” means that the corresponding radical, group, or moiety has no substituents.

The term “substituted” means that the corresponding radical, group, or moiety has one or more substituents. Where a radical has a plurality of substituents, and a selection of various substituents is specified, the substituents are selected independently of one another and do not need to be identical. Even though a radical has a plurality of a specific-designated substituent (e.g., Y₂ or YY), the expression of such substituent may differ from each other (e.g., methyl and ethyl). It shall be understood accordingly that a multiple substitution by any radical of the invention may involve identical or different radicals. Hence, if individual radicals occur several times within a compound, the radicals adopt the meanings indicated, independently of one another. In case of a multiple substitution, the radical could be alternatively designated with R′, R″, R′″, R″″ etc.

The terms “alkyl” or “A” refer to acyclic saturated or unsaturated hydrocarbon radicals, which may be branched or straight-chain and preferably have 1, 2, 3, 4, 5, 6, 7, 8, 9 or 10 carbon atoms, i.e., C₁-C₁₀-alkanyls. Examples of suitable alkyl radicals are methyl, ethyl, n-propyl, isopropyl, 1,1-, 1,2- or 2,2-dimethylpropyl, 1-ethylpropyl, 1-ethyl-1-methylpropyl, 1-ethyl-2-methylpropyl, 1,1,2- or 1,2,2-trimethylpropyl, n-butyl, isobutyl, sec-butyl, tert-butyl, 1-, 2- or 3-methylbutyl, 1,1-, 1,2-, 1,3-, 2,2-, 2,3- or 3,3-dimethylbutyl, 1- or 2-ethylbutyl, n-pentyl, iso-pentyl, neo-pentyl, tert-pentyl, 1-, 2-, 3- or -methyl-pentyl, n-hexyl, 2-hexyl, isohexyl, n-heptyl, n-octyl, n-nonyl, n-decyl, n-undecyl, n-dodecyl, n-tetradecyl, n-hexadecyl, n-octadecyl, n-icosanyl, and n-docosanyl.

In a preferred embodiment of the invention, A denotes unbranched or branched alkyl having 1-10 C atoms, in which 1-7 H atoms may be replaced independently from one another by Hal. More preferably, A denotes unbranched or branched alkyl having 1-6 C atoms, in which 1-4 atoms may be replaced independently from one another by Hal. In a most preferred embodiment of the invention, A denotes unbranched or branched alkyl having 1-4 C atoms, in which 1-3 H atoms can be replaced independently from one another by Hal. It is highly preferred that A denotes unbranched or branched alkyl having 1-4 C atoms, optionally in which 1-3 H atoms can be replaced independently from one another by F and/or Cl. Particularly preferred is C₁₋₄-alkyl. Such a C₁-4-alkyl radical is for example a methyl, ethyl, propyl, isopropyl, butyl, isobutyl, tert-butyl, sec-butyl, tert-butyl, fluoromethyl, difluoromethyl, trifluoromethyl, pentafluoroethyl, 1,1,1-trifluoroethyl or bromomethyl, especially methyl, ethyl, propyl or trifluoromethyl. Highly preferred is C₁₋₂-alkyl. It shall be understood that the respective denotation of A is independently of one another in any radical of the invention.

The terms “heterocycle” or “heterocyclyl” for the purposes of this invention refers to a mono- or polycyclic system of 3 to 14 ring atoms, preferably 4 to 10 ring atoms, more preferably 4 to 8 ring atoms, comprising carbon atoms and 1, 2, 3, 4 or 5 heteroatoms, which are identical or different, in particular nitrogen, oxygen and/or sulfur. The cyclic system may be saturated, mono- or poly-unsaturated, or aromatic. In the case of a cyclic system consisting of at least two rings the rings may be fused or spiro or otherwise connected. Such heterocyclyl radicals can be linked via any ring member. The term “heterocyclyl” also includes systems in which the heterocycle is part of a bi- or polycyclic saturated, partially unsaturated and/or aromatic system, such as where the heterocycle is fused to an aryl, cycloalkyl, heteroaryl or heterocyclyl group as defined herein via any desired and possible ring member of the heterocyclyl radical.

The compounds of the general formula (I) can be bonded via any possible ring member of the heterocyclyl radical. Examples of suitable heterocyclyl radicals are pyrrolidinyl, thiapyrrolidinyl, piperidinyl, piperazinyl, oxapiperazinyl, oxapiperidinyl, oxadiazolyl, tetrahydrofuryl, imidazolidinyl, thiazolidinyl, tetrahydropyranyl, morpholinyl, tetrahydrothiophenyl, and dihydropyranyl.

The term “heteroaryl” for the purposes of this invention refers to a 1-15, preferably 1-9, most preferably 5-, 6- or 7-membered mono- or polycyclic aromatic hydrocarbon radical which comprises at least 1, where appropriate also 2, 3, 4 or 5 heteroatoms, preferably nitrogen, oxygen and/or sulfur, where the heteroatoms are identical or different. Preferably, the number of nitrogen atoms is 0, 1, 2, 3 or 4, and that of the oxygen and sulfur atoms is independently from one another 0 or 1. The term “heteroaryl” also includes systems in which the aromatic cycle is part of a bi- or polycyclic saturated, partially unsaturated and/or aromatic system, such as where the aromatic cycle is fused to an aryl, cycloalkyl, heteroaryl or heterocyclyl group as defined herein via any desired and possible ring member of the heteroaryl radical. The compounds of the general formula (I) can be bonded via any possible ring member of the heteroaryl radical. Examples of suitable heteroaryl are pyrrolyl, thienyl, furyl, imidazolyl, thiazolyl, isothiazolyl, oxazolyl, oxadiazolyl, isoxazolyl, pyrazolyl, pyridinyl, pyrimidinyl, pyridazinyl, pyrazinyl, indolyl, quinolinyl, isoquinolinyl, imidazolyl, triazolyl, triazinyl, tetrazolyl, phthalazinyl, indazolyl, indolizinyl, quinoxalinyl, quinazolinyl, pteridinyl, carbazolyl, phenazinyl, phenoxazinyl, phenothiazinyl, and acridinyl.

It is preferred that heteroaryl in the realms of “Het” denotes an optionally substituted, saturated, unsaturated, or aromatic monocyclic 5-6-membered heterocycle having 2-5 C atoms and 1-3 N, O and/or S atoms. For example, Het can be substituted by at least one substituent selected from the group of A, Hal, OY, CN, COY, COOY, CONYY, NYCOY, NYCONYY, SO₂Y, SO₂NYY, NYSO₂Y, NYY, NO₂, OCN, SCN and SH. In a more preferred embodiment of the invention, Het denotes an unsaturated or aromatic monocyclic 5-6-membered heterocycle having 2-5 C atoms and 1-2 N atoms, which can be substituted by A or Hal. It is most preferred that Het denotes an unsaturated monocyclic 5-membered heterocycle having 3-4 C atoms and 1-2 N atoms, which can be monosubstituted by A. Highly preferred Het denotes 1-methylpyrazole.

The term “halogen”, “halogen atom”, “halogen substituent” or “Hal” for the purposes of this invention refers to one or, where appropriate, a plurality of fluorine (F, fluoro), bromine (Br, bromo), chlorine (Cl, chloro), or iodine (I, iodo) atoms. The designations “dihalogen”, “trihalogen” and “perhalogen” refer respectively to two, three and four substituents, where each substituent can be selected independently from the group consisting of fluorine, chlorine, bromine, and iodine. Halogen preferably means a fluorine (F), chlorine (Cl), or bromine (Br) atom. Fluorine and chlorine are more preferred, particularly when the halogens are substituted on an alkyl (haloalkyl) or alkoxy group (e.g. CF₃ and CF₃O). Most preferably, Hal denotes C. It shall be understood that the respective denotation of Hal is independently of one another in any radical of the invention.

In the present invention, W¹, W², W³, W⁴ denote independently from one another N or CH. In an embodiment of the present invention, W¹, W², W³, W⁴ denote independently from one another N or CH, with the proviso that at least one of W³ or W⁴ denotes N. In other words, either W³ or W⁴ denotes N while the respective other radical denotes N or CH.

In a preferred embodiment of the invention, W² denotes CH. In a preferred embodiment of the invention, W¹ denotes N. In a preferred embodiment of the invention, W³ denotes N. In a preferred embodiment of the invention, W⁴ denotes N.

In a preferred embodiment of the invention, W¹, W³ and/or W⁴ denote N. In a more preferred embodiment of the invention, W¹ denotes N. In a most preferred embodiment of the invention, W¹ and W³ denote N. In a highly preferred embodiment of the invention, W¹, W³, and W⁴ denote N.

In a more preferred embodiment of the invention, W² denotes CH, and/or W¹, W³ and/or W⁴ denote N. In a most preferred embodiment of the invention, W² denotes CH, and W¹ denotes N. In a highly preferred embodiment of the invention, W² denotes CH, and W¹ and W³ denote N. In a particularly highly preferred embodiment of the invention, W denotes CH, and W¹, W³ and W⁴ denote N.

The R¹ radical according to the present invention denotes NYSO₂Y or Y. It is a preferred embodiment of the R⁴ radical according to the present invention to be NASO₂A or H. More preferably, R¹ is NASO₂A.

The R², R⁴ radicals according to the present invention denote Y. It is a preferred embodiment of the R² radical according to the present invention to be H. It is a preferred embodiment of the R⁴ radical according to the present invention to be H.

The R³ radical according to the present invention denotes Y or Hal. It is a preferred embodiment of the R³ radical according to the present invention to be H or Hal. More preferably, R³ is H.

In another preferred embodiment of the invention, R², R³ and/or R⁴ denote H.

In another aspect of the invention, the R², R³ radicals according to the present invention together also denote —(CY)₂- or —(CR⁷)—(CY)₂—. Preferably, R², R³ together denote —(CY)₂- or —(CR⁷)—(CY)₂—, with the proviso that W⁴ denotes N. More preferably, R², R³ together denote —(CY)₂—, most preferably —(CA)₂-, highly preferably in each case with the proviso that W⁴ denotes N. In other words, highly preferably, R², R³ together denote —(CY)₂- with the proviso that W⁴ denotes N.

The R⁵, R⁶ radicals according to the present invention denote independently from one another Hal, Y or Het. It is a preferred embodiment of the R⁵ radical according to the present invention to be Hal or A. More preferably, R⁵ is Hal. It is a preferred embodiment of the R⁶ radical according to the present invention to be Hal or A. More preferably, R⁶ is Hal.

The R⁷ radical according to the present invention denotes Y or ═O. It is a preferred embodiment of the R radical according to the present invention to be ═O.

The m index according to the present invention denotes 0, 1, 2 or 3, preferably 0 or 1, more preferably 0.

It is another preferred embodiment that the R⁵, R⁶ radicals according to the present invention denote independently from one another Hal or A, and/or m denotes 0 or 1.

In an aspect of the invention, Y denotes H or A. It shall be understood that the respective denotation of Y is independently of one another in any radical of the invention.

Accordingly, the subject-matter of the invention relates to compounds of formula (I), in which at least one of the above-identified radicals has any meaning, particularly realize any aspect or preferred embodiment, as described above. Radicals, which are not explicitly specified in the context of any aspect or embodiment of formula (I), sub-formulae thereof or other radicals thereto, shall be construed to represent any respective denotations according to formula (I) as disclosed hereunder for solving the problem of the invention. That means, the above-identified radicals may adopt all designated meanings as each described in the prior or following course of the present specification, irrespective of the context to be found, including, but not limited to, any preferred embodiments. It shall be particularly understood that any embodiment of a certain radical can be combined with any embodiment of one or more other radicals.

In another preferred embodiment of the present invention, a compound of sub-formula (I-A) is provided

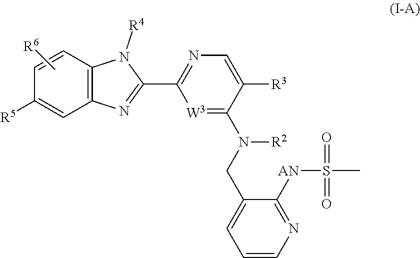

wherein
R⁵, R⁶ denote independently from one another Hal or A, and
W³, R², R³, R⁴ and A have the meaning as defined above;
and/or a physiologically acceptable salt thereof.

The prior teaching of the present specification concerning the compounds of formula (I), including any radical definition and preferred embodiment thereof, is valid and applicable without restrictions to the compounds according to sub-formula (I-A) and the physiologically acceptable salt, if appropriate.

Highly preferred embodiments are those compounds of formulae (I) and (I-A) as listed below:

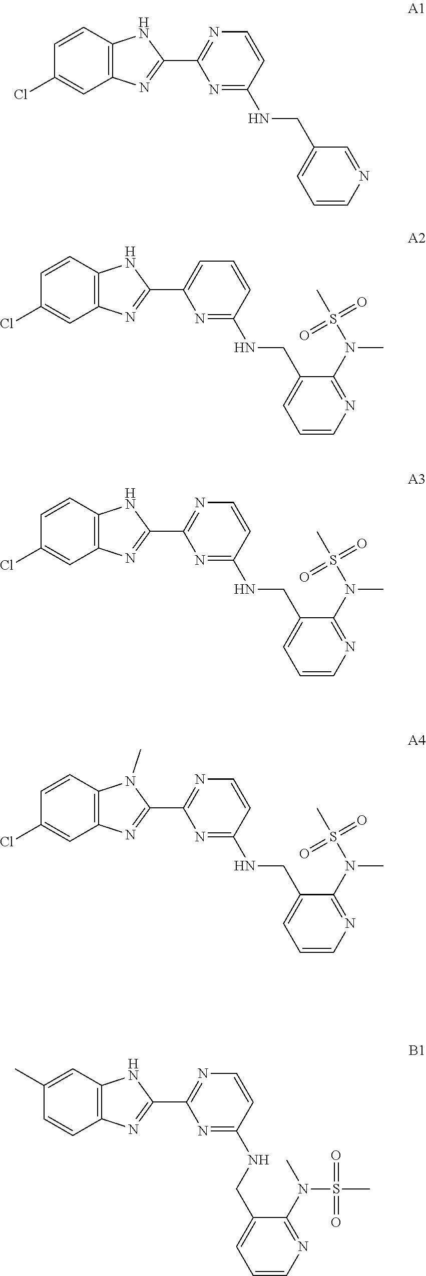

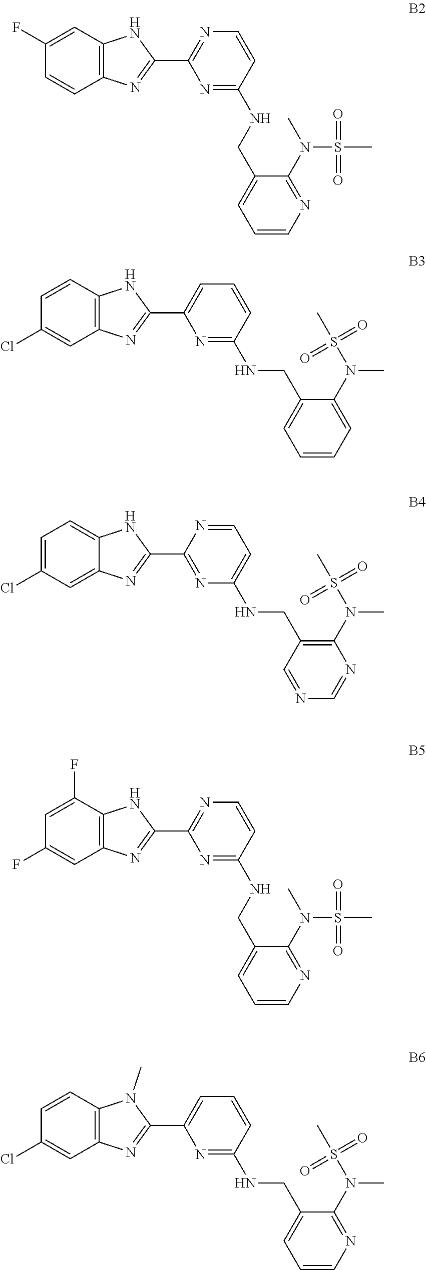 [SMILES: CN(c1ncccc1CNc1ccnc(-c2nc3ccc(F)cc3[nH]2)n1)S(C)(=O)=O ; CN(c1ccccc1CNc1cccc(-c2nc3cc(Cl)ccc3[nH]2)n1)S(C)(=O)=O ; CN(c1ncncc1CNc1ccnc(-c2nc3cc(Cl)ccc3[nH]2)n1)S(C)(=O)=O ; CN(c1ncccc1CNc1ccnc(-c2nc3cc(F)cc(F)c3[nH]2)n1)S(C)(=O)=O ; CN(c1ncccc1CNc1cccc(-c2nc3cc(Cl)ccc3n2C)n1)S(C)(=O)=O]

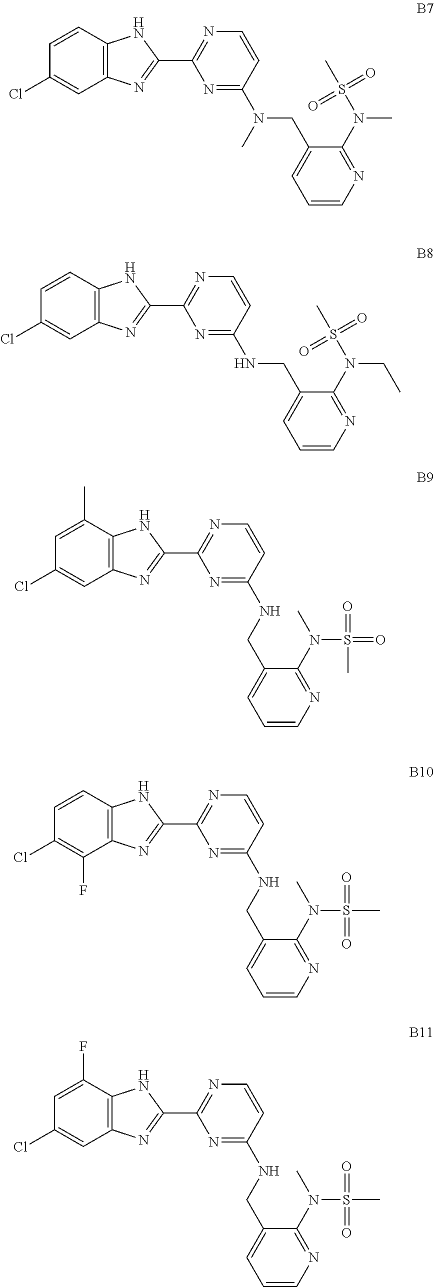 [SMILES: CN(Cc1cccnc1N(C)S(C)(=O)=O)c1ccnc(-c2nc3cc(Cl)ccc3[nH]2)n1 ; CCN(c1ncccc1CNc1ccnc(-c2nc3cc(Cl)ccc3[nH]2)n1)S(C)(=O)=O ; Cc1cc(Cl)cc2nc(-c3nccc(NCc4cccnc4N(C)S(C)(=O)=O)n3)[nH]c12 ; CN(c1ncccc1CNc1ccnc(-c2nc3c(F)c(Cl)ccc3[nH]2)n1)S(C)(=O)=O ; CN(c1ncccc1CNc1ccnc(-c2nc3cc(Cl)cc(F)c3[nH]2)n1)S(C)(=O)=O]

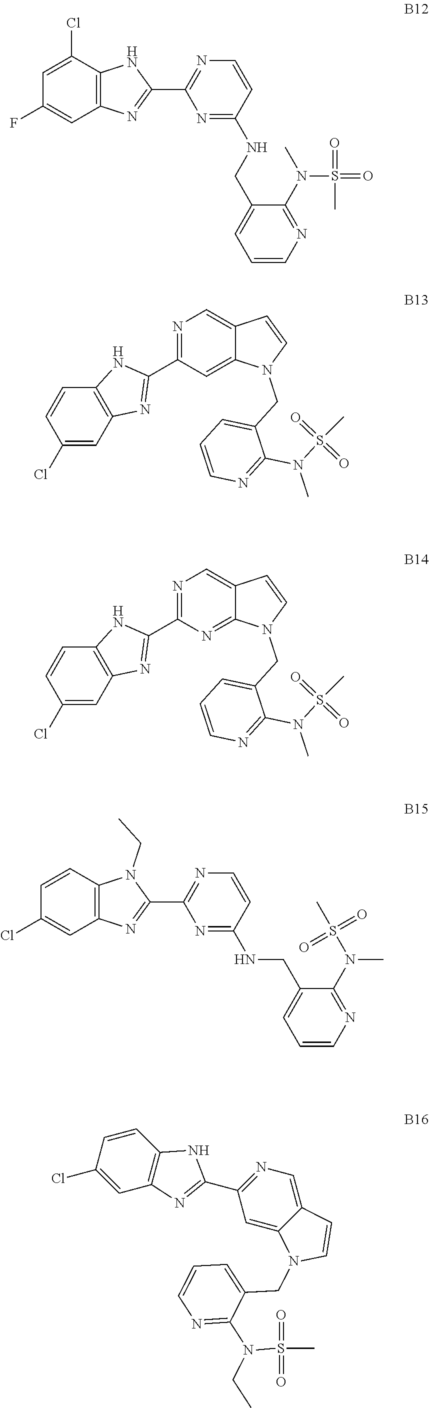 [SMILES: CN(c1ncccc1CNc1ccnc(-c2nc3cc(F)cc(Cl)c3[nH]2)n1)S(C)(=O)=O ; CN(c1ncccc1Cn1ccc2cnc(-c3nc4cc(Cl)ccc4[nH]3)cc21)S(C)(=O)=O ; CN(c1ncccc1Cn1ccc2cnc(-c3nc4cc(Cl)ccc4[nH]3)nc21)S(C)(=O)=O ; CCn1c(-c2nccc(NCc3cccnc3N(C)S(C)(=O)=O)n2)nc2cc(Cl)ccc21 ; CCN(c1ncccc1Cn1ccc2cnc(-c3nc4cc(Cl)ccc4[nH]3)cc21)S(C)(=O)=O]

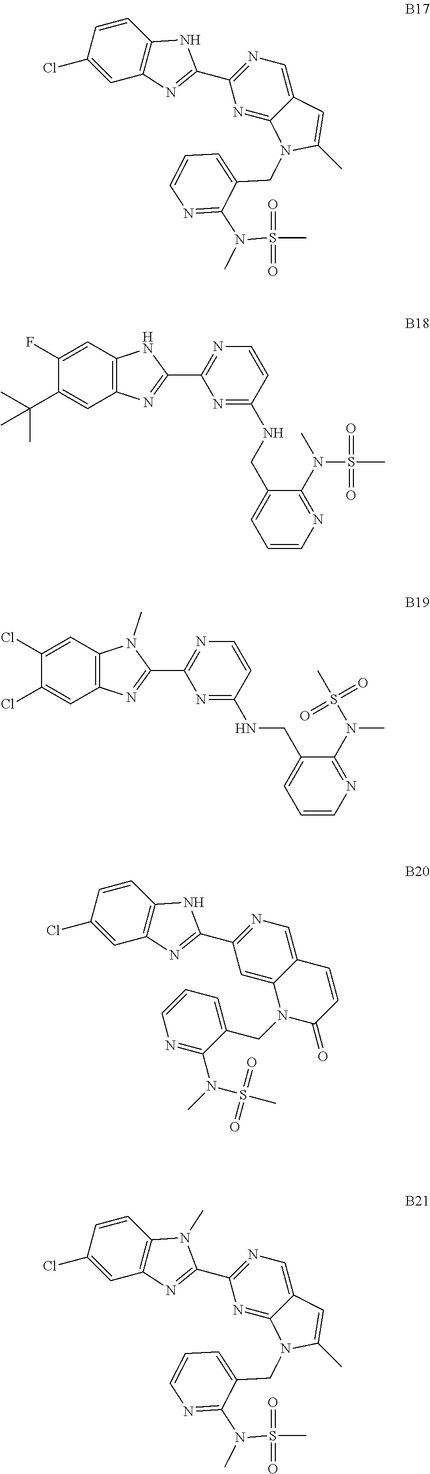

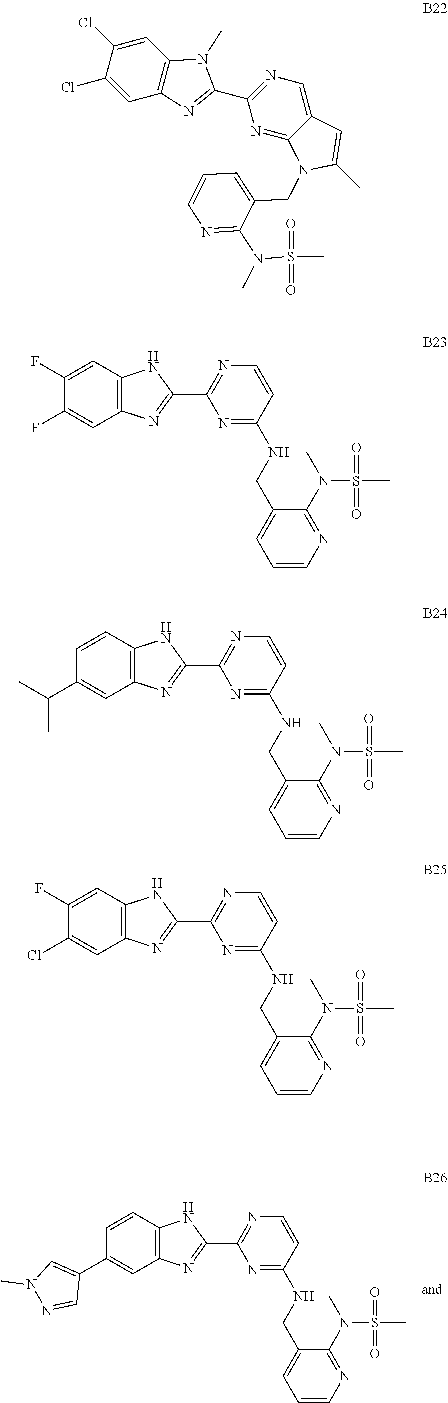

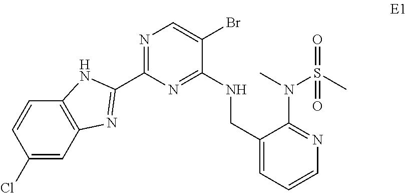 [SMILES: CN(c1ncccc1CNc1nc(-c2nc3cc(Cl)ccc3[nH]2)ncc1Br)S(C)(=O)=O]

and/or a physiologically acceptable salt thereof.

The compounds according to formula (I) and the starting materials for its preparation, respectively, are produced by methods known per se, as described in the literature (e.g., in standard books, such as Houben-Weyl, Methods of Organic Chemistry), i.e., under reaction conditions that are known and suitable for said reactions.

Use can also be made of variants that are known per se but are not mentioned in greater detail herein. If desired, the starting materials can also be formed in-situ by leaving them in the urn-isolated status in the crude reaction mixture, but immediately converting them further into the compound according to the invention. It is also possible to carry out the reaction stepwise.

The reaction is generally carried out in an inert solvent. Suitable inert solvents are, e.g., hydrocarbons, such as hexane, petroleum ether, benzene, toluene, or xylene; chlorinated hydrocarbons, such as trichloroethylene, 1,2-dichloroethane, carbon tetrachloride, chloroform, or dichloromethane; alcohols, such as methanol, ethanol, isopropanol, n-propanol, n-butanol, or tert-butanol; ethers, such as diethyl ether, diisopropyl ether, tetrahydrofuran (THF), or dioxane; glycol ethers, such as ethylene glycol monomethyl or monoethyl ether, ethylene glycol dimethyl ether (diglyme); ketones, such as acetone or butanone; amides, such as acetamide, dimethylacetamide, or dimethylformamide (DMF); nitriles, such as acetonitrile; sulfoxides, such as dimethyl sulfoxide (DMSO); carbon disulfide; carboxylic acids, such as formic acid, acetic acid or trifluoroacetic acid (TFA); nitro compounds, such as nitromethane or nitrobenzene; esters, such as ethyl acetate, or mixtures of said solvents. Particular preference is given to DMF, TFA, H₂O, THF, tert.-butanol, tert.-amylalcohol, triethylamine, or dioxane.

Depending on the conditions used, the reaction time is between a few minutes and 14 days, the reaction temperature is between about −30° C. and 140° C., normally between −10° C. and 130° C., preferably between 0° C. and 100° C.

The present invention also relates to a process for manufacturing a compound of formula (I) comprising the steps of:
(a) reacting a compound of formula (II)

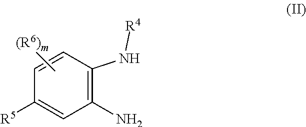

wherein R⁴, R⁷), R⁶ and m have the meaning as defined above,
with a compound of formula (III)

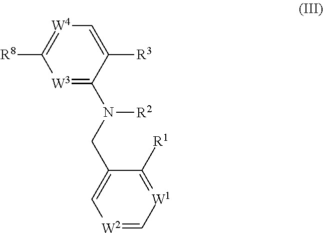

wherein
R⁸ denotes CN, COOH or Hal; and
W¹, W², W³, W⁴, R¹, R² and R³ have the meaning as defined above,
to yield a compound of formula (I)

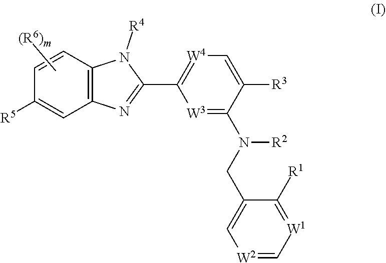

wherein W¹, W², W³, W⁴, R¹, R², R³, R⁴, R⁵, R⁶ and m have the meaning as defined above,
and optionally
(b) converting a base or an acid of the compound of formula (I) into a salt thereof.

The compounds of formula (I) are accessible via the route above. The starting materials, including the compounds of formulae (II) and (Ill) are usually known to the skilled artisan, or they can be easily prepared by known methods. Accordingly, any compounds of formulae (II) and (Ill) can be purified, provided as intermediate product, and used as starting material for the preparation of compounds of formula (I).

In the final step of the processes above, a salt of the compounds according to formula (I) is optionally provided. The said compounds according to the invention can be used in their final non-salt form. On the other hand, the present invention also encompasses the use of these compounds in the form of their pharmaceutically acceptable salts, which can be derived from various organic and inorganic acids and bases by procedures known in the art. Pharmaceutically acceptable salt forms of the compounds according to the invention are for the most part prepared by conventional methods. If the compound according to the invention contains a carboxyl group, one of its suitable salts can be formed by the reaction of the compound with a suitable base to give the corresponding base-addition salt. Such bases are, for example, alkali metal hydroxides, including potassium hydroxide, sodium hydroxide, and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine, and N-methylglutamine. The aluminum salts of the compounds according to the invention are likewise included. In most case of the compounds according to the invention, it is preferred that acid-addition salts are formed by treating these compounds with pharmaceutically acceptable organic and inorganic acids, for example hydrogen halides, such as hydrogen chloride, hydrogen bromide, or hydrogen iodide, other mineral acids and corresponding salts thereof, such as sulfate, nitrate, or phosphate and the like, and alkyl- and monoarylsulfonates, such as ethanesulfonate, toluenesulfonate, and benzenesulfonate, and other organic acids and corresponding salts thereof, such as acetate, trifluoroacetate, tartrate, maleate, succinate, citrate, benzoate, salicylate, ascorbate, and the like. Accordingly, pharmaceutically acceptable acid-addition salts of the compounds according to the invention include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, and phthalate, but this does not represent a restriction.

With regard to that stated above, it can be seen that the expressions “pharmaceutically acceptable salt” and “physiologically acceptable salt”, which are used interchangeable herein, in the present connection are taken to mean an active ingredient which comprises a compound according to the invention in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Object of the present invention is also the use of compounds according to formula (I) and/or physiologically acceptable salts thereof for modulating and preferably inhibiting SRPK activity.

The term “modulation” denotes any change in SRPK-mediated signal transduction, which is based on the action of the specific inventive compounds capable to interact with the SRPK target in such a manner that makes recognition, binding, and inhibition possible.

The term “inhibition” denotes any reduction in SRPK activity, which is based on the action of the specific compounds of formula (I), which are capable to interact with the target SRPK in such a manner that makes recognition, binding, and blocking possible. The compounds are characterized by such an appreciable affinity to SRPK, which ensures a reliable binding and blocking of SRPK activity. Preferably, the compounds are SRPK-specific to guarantee an exclusive and directed recognition of the SRPK target. In an embodiment of the invention, the compounds of formula (I) are bi-specific to guarantee an exclusive and directed recognition of two targets selected from the group of SRPK1, SRPK2 and SRPK3. In another embodiment of the invention, the compounds of formula (I) are tri-specific to guarantee an exclusive and directed recognition of the targets SRPK1, SRPK2 and SRPK3. In another embodiment of the present invention, the compound of formula (I) and/or a physiologically acceptable salt thereof are SRPK inhibitors which do not show cross-reactivity with CLK and/or DYRK proteins.

In the context of the present invention, the term “recognition”—without being limited thereto—relates to any type of interaction between the specific compounds and the target, particularly covalent or non-covalent binding or association, such as a covalent bond, hydrophobic/hydrophilic interactions, van der Waals forces, ion pairs, hydrogen bonds, ligand-receptor interactions, and the like. Such association may also encompass the presence of other molecules such as peptides, proteins, or nucleotide sequences. The present interaction is characterized by high affinity, high selectivity, and minimal or even lacking cross-reactivity to other target molecules to exclude unhealthy and harmful impacts to the treated subject.

A preferred object of the present invention relates to a method for inhibiting SRPK, wherein a system capable of expressing SRPK, preferably expressing the SRPK, is contacted with at least one compound of formula (I) according to the invention and/or a physiologically acceptable salt thereof, under conditions such that said SRPK is inhibited. A cellular system is preferred in the scope of the invention. The cellular system is defined to be any subject provided that the subject comprises cells. Hence, the cellular system can be selected from the group of single cells, cell cultures, tissues, organs, and animals. The method for inhibiting SRPK is preferably performed in-vitro. The prior teaching of the present specification concerning the compounds of formula (I), including any preferred embodiment thereof, is valid and applicable without restrictions to the compounds according to formula (I) and their salts when used in the method for inhibiting SRPK.

The compounds according to the invention preferably exhibit an advantageous biological activity, which is easily demonstrated in cell culture-based assays, for example assays as described herein or in prior art. In such assays, the compounds according to the invention preferably exhibit and cause an inhibiting effect. The compounds of the invention exhibit IC₅₀ values in the range of 1 nM to 25 μM. It is preferred that the compounds of the invention have an activity, as expressed by an IC₅₀ standard, of 2.5 μM or less, preferably 1 μM or less, more preferably 0.5 μM or less, most preferably less than 0.05 μM.

The method of the invention can be performed either in-vitro or in-vivo. The susceptibility of a particular cell to treatment with the compounds according to the invention can be particularly determined by in-vitro tests, whether during research or clinical application. Typically, a culture of the cell is combined with a compound according to the invention at various concentrations for a period of time which is sufficient to allow the active agents to modulate SRPK activity, usually between about one hour and one week. In-vitro treatment can be carried out using cultivated cells from a biopsy sample or cell line. In a preferred aspect of the invention, a follicle cell is stimulated for maturation. The viable cells remaining after the treatment are counted and further processed.

The host or patient can belong to any mammalian species, for example a primate species, particularly humans; rodents, including mice, rats, and hamsters; rabbits; horses, cows, dogs, cats, etc. Animal models are of interest for experimental investigations, providing a model for treatment of human disease.

For identification of a signal transduction pathway and for detection of interactions between various signal transduction pathways, various scientists have developed suitable models or model systems, e.g., cell culture models and models of transgenic animals. For the determination of certain stages in the signal transduction cascade, interacting compounds can be utilized to modulate the signal. The compounds according to the invention can also be used as reagents for testing SRPK-dependent signal transduction pathways in animals and/or cell culture models or in the clinical diseases mentioned in this application.

The use according to the previous paragraphs of the specification may be either performed in-vitro or in-vivo models. The modulation can be monitored by the techniques described in the present specification. The in-vitro use is preferably applied to samples of humans suffering from hyperproliferative disorders. Testing of several specific compounds and/or derivatives thereof makes the selection of that active ingredient possible that is best suited for the treatment of the human subject. The in-vivo dose rate of the chosen derivative is advantageously pre-adjusted to the SRPK susceptibility and/or severity of disease of the respective subject with regard to the in-vitro data. Therefore, the therapeutic efficacy is remarkably enhanced. Moreover, the subsequent teaching of the present specification concerning the use of the compounds according to formula (I) and its pharmaceutically acceptable salts for the production of a medicament for the prophylactic or therapeutic treatment and/or monitoring is considered as valid and applicable without restrictions to the use of the compound for the modulation of SRPK activity, if appropriate.

Accordingly, the compounds according to the invention are useful in the prophylaxis and/or treatment of diseases that are dependent on the said signaling pathways by interaction with one or more of the said signaling pathways. The present invention therefore relates to compounds according to the invention as modulators, preferably inhibitors, of the signaling pathways described herein. In particular, the invention relates to the use of compounds according to the invention for the preparation of a medicament for the treatment of hyperproliferative diseases related to the hyperactivity of SRPK as well as diseases modulated by the SRPK cascade in mammals, or disorders mediated by aberrant proliferation, such as cancer and inflammation.

The invention furthermore relates to a medicament comprising at least one compound according to the invention and/or pharmaceutically usable derivatives, salts, solvates and stereoisomers thereof, including mixtures thereof in all ratios. Preferably, the invention relates to a medicament comprising at least one compound according to the invention and/or physiologically acceptable salts thereof.

A “medicament” in the meaning of the invention is any agent in the field of medicine, which comprises one or more compounds of formula (I) or preparations thereof (e.g., a pharmaceutical composition or pharmaceutical formulation) and can be used in prophylaxis, therapy, follow-up or aftercare of patients who suffer from diseases, which are associated with SRPK activity, in such a way that a pathogenic modification of their overall condition or of the condition of particular regions of the organism could establish at least temporarily.

The invention also relates to a pharmaceutical composition comprising as active ingredient at least one compound of formula (I) according to the invention and/or physiologically acceptable salts thereof together with pharmaceutically tolerable adjuvants and/or excipients. It shall be understood that the compound of the invention is provided in an effective amount.

In the meaning of the invention, an “adjuvant” denotes every substance that enables, intensifies, or modifies a specific response against the active ingredient of the invention if administered simultaneously, contemporarily, or sequentially. Known adjuvants for injection solutions are, for example, aluminum compositions, such as aluminum hydroxide or aluminum phosphate; saponins, such as QS21, muramyldipeptide, or muramyltripeptide; proteins, such as gamma-interferon or TNF; M59, squalen or polyols.

Furthermore, the active ingredient may be administered alone or in combination with other treatments. A synergistic effect may be achieved by using more than one compound in the pharmaceutical composition, i.e., the compound of formula (I) is combined with at least another agent as active ingredient, which is either another compound of formula (I) or a compound of different structural scaffold. The active ingredients can be used either simultaneously or sequentially. The invention also relates to a compound or pharmaceutical composition for inhibiting abnormal cell growth or cancer in a mammal which comprises an amount of a compound of the present invention, or a pharmaceutically acceptable salt or solvate or prodrug thereof, in combination with an amount of another anti-cancer therapeutic, wherein the amounts of the compound, salt, solvate, or prodrug, and of the chemotherapeutic are together effective in inhibiting abnormal cell growth or cancer. The present compounds are suitable for combination with known anti-cancer agents.

Many oncology therapeutics are presently known in the art. In a preferred embodiment, the other active pharmaceutical ingredient is an anti-cancer therapeutic that is a chemotherapeutic selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. In another preferred embodiment of the invention, the anti-cancer therapeutic is an antibody selected from the group consisting of bevacizumab, CD40-specific antibodies, chTNT-1/B, denosumab, zanolimumab, IGF1R-specific antibodies, lintuzumab, edrecolomab, WX G250, rituximab, ticilimumab, trastuzumab, and cetuximab. In yet another preferred embodiment of the invention, the anti-cancer therapeutic is an inhibitor of another protein kinase, such as Akt, Axl, dyrk2, epha2, fgfr3, igf1r, IKK2, JNK3, Vegfr1, Vegfr2, Vegfr3 (also known as Flt-4), KDR, MEK, MET, PIk1, RSK1, Src, TrkA, Zap70, cKit, bRaf, EGFR, Jak2, PI3K, NPM-Alk, c-Abl, BTK, FAK, PDGFR, TAK1, LimK, Flt-3, PDK1, and Erk. Further anti-cancer agents are known to those of skill in the art and are useful with the compounds of the present invention.

The invention also relates to a set (kit) consisting of separate packs of an effective amount of a compound according to the invention and/or pharmaceutically acceptable salts, derivatives, solvates and stereoisomers thereof, including mixtures thereof in all ratios, and an effective amount of a further medicament active ingredient. The set comprises suitable containers, such as boxes, individual bottles, bags, or ampoules. The set may, for example, comprise separate ampoules, each containing an effective amount of a compound according to the invention and/or pharmaceutically acceptable salts, derivatives, solvates and stereoisomers thereof, including mixtures thereof in all ratios, and an effective amount of a further medicament active ingredient in dissolved or lyophilized form.

Pharmaceutical formulations can be adapted for administration via any desired suitable method, for example, by oral (including buccal or sublingual), rectal, nasal, topical (including buccal, sublingual, or transdermal), vaginal or parenteral (including subcutaneous, intramuscular, intravenous, or intradermal) methods. Such formulations can be prepared using all processes known in the pharmaceutical art by, for example, combining the active ingredient with the excipient(s) or adjuvant(s).

The pharmaceutical composition of the invention is produced in a known way using common solid or liquid carriers, diluents and/or additives and usual adjuvants for pharmaceutical engineering and with an appropriate dosage. The amount of excipient material that is combined with the active ingredient to produce a single dosage form varies depending upon the host treated and the mode of administration. Suitable excipients include organic or inorganic substances that are suitable for the different routes of administration, such as enteral (e.g., oral), parenteral or topical application, and which do not react with compounds of formula (I) or salts thereof. Examples of suitable excipients are water, vegetable oils, benzyl alcohols, alkylene glycols, polyethylene glycols, glycerol triacetate, gelatin, carbohydrates, e.g., lactose or starch, magnesium stearate, talc, and petroleum jelly.

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multi-dose containers, for example, sealed ampoules and vials, and stored in freeze-dried (lyophilized) state, so that only the addition of the sterile carrier liquid, for example water for injection purposes, immediately before use is necessary. Injection solutions and suspensions prepared in accordance with the recipe can be prepared from sterile powders, granules, and tablets.

It goes without saying that, in addition to the above particularly mentioned constituents, the formulations may also comprise other agents usual in the art with respect to the particular type of formulation; thus, for example, formulations which are suitable for oral administration may comprise flavors.

In a preferred embodiment of the present invention, the pharmaceutical composition is adapted for oral administration. The preparations can be sterilized and/or can comprise auxiliaries, such as carrier proteins (e.g., serum albumin), lubricants, preservatives, stabilizers, fillers, chelating agents, antioxidants, solvents, bonding agents, suspending agents, wetting agents, emulsifiers, salts (for influencing the osmotic pressure), buffer substances, colorants, flavorings, and one or more further active substances, for example, one or more vitamins. Additives are well known in the art, and they are used in a variety of formulations.

The invention also relates to a pharmaceutical composition comprising as active pharmaceutical ingredient at least one compound of formula (I) according to the invention and/or physiologically acceptable salts thereof together with pharmaceutically tolerable adjuvants, optionally in combination with at least another active pharmaceutical ingredient. Both active pharmaceutical ingredients are particularly provided in effective amounts. The prior teaching of the present specification concerning administration route or combination product is valid and applicable without restrictions to the combination of both features, if appropriate.

The terms “effective amount” or “effective dose” or “dose” are interchangeably used herein and denote an amount of the pharmaceutical compound having a prophylactically or therapeutically relevant effect on a disease or pathological conditions, i.e., which causes in a tissue, system, animal or human such a biological or medical response which is sought or desired, for example, by a researcher or physician.

A “prophylactic effect” reduces the likelihood of developing a disease or even prevents the onset of a disease. A “therapeutically relevant effect” relieves to some extent one or more symptoms of a disease or returns to normality either partially or completely one or more physiological or biochemical parameters associated with or causative of the disease or pathological conditions. In addition, the expression “therapeutically effective amount” denotes an amount which, compared with a corresponding subject who has not received this amount, has the following consequence: improved treatment, healing, prevention or elimination of a disease, syndrome, condition, complaint, disorder or side-effects, or also the reduction in the advance of a disease, complaint or disorder. The expression “therapeutically effective amount” also encompasses the amounts which are effective for increasing normal physiological function.

The respective dose or dosage range for administering the pharmaceutical composition according to the invention is sufficiently high to achieve the desired prophylactic or therapeutic effect of reducing symptoms of the above-identified diseases, such as cancer and inflammation. It will be understood that the specific dose level, frequency and period of administration to any particular human will depend upon a variety of factors including the activity of the specific compound employed, the age, body weight, general state of health, gender, diet, time and route of administration, rate of excretion, drug combination, and the severity of the particular disease to which the specific therapy is applied. Using well-known means and methods, the exact dose can be determined by one of skill in the art as a matter of routine experimentation. The prior teaching of the present specification is valid and applicable without restrictions to the pharmaceutical composition comprising the compounds of formula (I), if appropriate.

Pharmaceutical formulations can be administered in the form of dosage units which comprise a predetermined amount of active ingredient per dosage unit. The concentration of the prophylactically or therapeutically active ingredient in the formulation may vary from about 0.1 to 100 wt %. Preferably, the compound of formula (I) or the pharmaceutically acceptable salts thereof are administered in doses of approximately 0.5 to 1000 mg, more preferably between 1 and 700 mg, most preferably 5 and 100 mg per dose unit. Generally, such a dose range is appropriate for total daily incorporation. In other terms, the daily dose is preferably between approximately 0.02 and 100 mg/kg of body weight. The specific dose for each patient depends, however, on a wide variety of factors as already described in the present specification (e.g., depending on the condition treated, the method of administration and the age, weight, and condition of the patient). Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Although a therapeutically effective amount of a compound according to the invention has to be ultimately determined by the treating doctor or vet by considering a number of factors (e.g., the age and weight of the animal, the precise condition that requires treatment, severity of condition, the nature of the formulation and the method of administration), an effective amount of a compound according to the invention for the treatment of neoplastic growth, for example colon or breast carcinoma, is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as a single dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound according to the invention per se. It can be assumed that similar doses are suitable for the treatment of other conditions mentioned above.

The pharmaceutical composition of the invention can be employed as medicament in human and veterinary medicine. According to the invention, the compounds of formula (I) and/or physiologically salts thereof are suited for the prophylactic or therapeutic treatment and/or monitoring of diseases that are caused, mediated and/or propagated by SRPK activity. It is preferred that the diseases are selected from the group of hyperproliferative disorders, cancer, metastases, tumors, angiogenesis disorders, tumor angiogenesis, benign hyperplasia, hemangioma, glioma, melanoma, Kaposi's sarcoma, prostate diseases related to vasculogenesis or angiogenesis, inflammation, pancreatitis, retinopathy, retinopathy of prematurity, diabetic retinopathy, diabetes, pain, restenosis, psoriasis, eczema, scleroderma and age-related macular degeneration. It shall be understood that the host of the compound is included in the present scope of protection according to the present invention.

Particular preference is given to the treatment of cancer, such as brain, lung, colon, epidermoid, squamous cell, bladder, gastric, pancreatic, breast, head, neck, renal, kidney, liver, ovarian, prostate, colorectal, uterine, rectal, oesophageal, testicular, gynecological or thyroid cancer, or melanoma; hematologic malignancies, such as acute myelogenous leukemia, multiple myeloma, chronic myelogneous leukemia, or myeloid cell leukemia: glioma; Kaposi's sarcoma; or any other type of solid or liquid tumors. More preferably, the cancer to be treated is chosen from breast, colorectal, lung, prostate or pancreatic cancer, or glioblastoma.

Further preference is given to treatment of a disease related to vasculogenesis or angiogenesis in a mammal, which comprises the administration of a therapeutically effective amount of a compound of the present invention, or a pharmaceutically acceptable salt, prodrug or hydrate thereof, and a pharmaceutically acceptable carrier. In one embodiment, the compound or pharmaceutical composition of the invention is for treating a disease selected from the group consisting of tumor angiogenesis; chronic inflammatory disease, such as rheumatoid arthritis, inflammatory bowel disease, or atherosclerosis; skin diseases, such as psoriasis, eczema, or scleroderma; metabolic diseases, such as diabetes, obesity, metabolic syndrome, insulin resistance, hyperglycemia, hyperaminoacidemia, hyperlipidmia, diabetic retinopathy, or retinopathy of prematurity; and age-related macular degeneration.

The invention also relates to the use of compounds according to formula (I) and/or physiologically acceptable salts thereof for the prophylactic or therapeutic treatment and/or monitoring of diseases that are caused, mediated and/or propagated by SRPK activity. Furthermore, the invention relates to the use of compounds according to formula (I) and/or physiologically acceptable salts thereof for the production of a medicament for the prophylactic or therapeutic treatment and/or monitoring of diseases that are caused, mediated and/or propagated by SRPK activity. Compounds of formula (I) and/or a physiologically acceptable salt thereof can furthermore be employed as intermediate for the preparation of further medicament active ingredients. The medicament is preferably prepared in a non-chemical manner, e.g., by combining the active ingredient with at least one solid, fluid and/or semi-fluid carrier or excipient, and optionally in conjunction with a single or more other active substances in an appropriate dosage form.

Another object of the present invention are compounds of formula (I) according to the invention and/or physiologically acceptable salts thereof for use in the prophylactic or therapeutic treatment and/or monitoring of diseases that are caused, mediated and/or propagated by SRPK activity. Another preferred object of the invention concerns compounds of formula (I) according to the invention and/or physiologically acceptable salts thereof for use in the prophylactic or therapeutic treatment and/or monitoring of hyperproliferative disorders. The prior teaching of the present specification concerning the compounds of formula (I), including any preferred embodiment thereof, is valid and applicable without restrictions to the compounds according to formula (I) and their salts for use in the prophylactic or therapeutic treatment and/or monitoring of hyperproliferative disorders.

The compounds of formula (I) according to the invention can be administered before or following an onset of disease once or several times acting as therapy. The above-identified compounds and medical products of the inventive use are particularly used for therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the compounds are administered in distinct intervals, e.g., to booster the response and eradicate the pathogens and/or symptoms of the disease completely. Either the identical compound or different compounds can be applied. The medicament can also be used to reducing the likelihood of developing a disorder or even prevent the initiation of disorders associated with SRPK activity in advance or to treat the arising and continuing symptoms. The disorders as concerned by the invention are preferably hyperproliferative disorders.

In the meaning of the invention, prophylactic treatment is advisable if the subject possesses any preconditions for the above-identified physiological or pathological conditions, such as a familial disposition, a genetic defect, or a previously passed disease.

It is another object of the invention to provide a method for treating diseases that are caused, mediated and/or propagated by SRPK activity, wherein at least one compound of formula (I) according to the invention and/or a physiologically acceptable salt thereof is administered to a mammal in need of such treatment. It is another preferred object of the invention to provide a method for treating hyperproliferative disorders, wherein at least one compound of formula (I) according to the invention and/or a physiologically acceptable salt thereof is administered to a mammal in need of such treatment. The compound is preferably provided in an effective amount as defined above. The preferred treatment is an oral administration.

In another preferred aspect, the method for treating cancer in a mammal comprises administering to the mammal an amount of a compound of the present invention in combination with radiation therapy, wherein the amount of the compound is in combination with the radiation therapy effective in treating cancer in the mammal. Techniques for administering radiation therapy are known in the art, and these techniques can be used in the combination therapy described herein. The amount and administration of a compound of the invention in this combination therapy can be determined according to the means for ascertaining effective amounts, doses and routes of such compounds as described herein. It is believed that the compounds of the present invention can render abnormal cells more sensitive to treatment with radiation for purposes of killing and/or inhibiting the growth of such cells. Accordingly, this invention relates to a method for sensitizing abnormal cells in a mammal to treatment with radiation which comprises administering to the mammal an amount of a compound of the present invention which amount is effective in sensitizing abnormal cells to treatment with radiation.

It is still another aspect of the invention to provide a method for inhibiting abnormal cell growth in a mammal which comprises an amount of a compound of the present invention or an isotopically-labeled derivative thereof, and an amount of one or more substances selected from anti-angiogenesis agents, signal transduction inhibitors, and anti-proliferative agents.

The prior teaching of the invention and its embodiments is valid and applicable without restrictions to the methods of treatment, if appropriate.

In the scope of the present invention, novel SRPK-inhibiting compounds of formula (I) are provided for the first time. The invention comprises the use of compounds of formula (I) in the regulation, modulation and/or inhibition of SRPK. The compounds of the invention can be advantageously applied as research tool, for diagnosis and/or in treatment of any disorders that are responsive to SRPK signaling and inhibition.

For example, the compounds of the invention are useful in-vitro as unique tools for understanding the biological role of SRPK, including the evaluation of the many factors thought to influence, and be influenced by, the production of SRPK. The present compounds are also useful in the development of other compounds that interact with SRPK since the present compounds provide important structure-activity relationship (SAR) information that facilitate that development.

The compounds of the invention are potent, selective, and orally bioavailable SRPK inhibitors that address the unmet medical need for several conditions, particularly cancer and inflammation, with respect to the progressive features of the diseases. Medicaments and pharmaceutical compositions containing said compounds and the use of said compounds to treat SRPK-mediated conditions is a promising, novel approach for a broad spectrum of therapies causing a direct and immediate improvement in the state of health, whether in man and animal. The impact is of special benefit to efficiently combat hyperproliferative disorders, either alone or in combination with other treatments.

Due to the surprisingly appreciable inhibitory activity on SRPK, the compounds of the invention can be advantageously administered at lower doses compared to other less potent or selective inhibitors of prior art while still achieving equivalent or even superior desired biological effects. In addition, such a dose reduction advantageously leads to less or even no medicinal adverse effects. Moreover, the compounds of formula (I), their salts, isomers, tautomers, enantiomeric forms, diastereomers, racemates, derivatives, prodrugs and/or metabolites are characterized by a high specificity and stability, low manufacturing costs and convenient handling. These features form the basis for a reproducible action, wherein the lack of cross-reactivity is included, and for a reliable and safe interaction with the target structure.

All the references cited herein are incorporated by reference in the disclosure of the invention hereby.

It is to be understood that this invention is not limited to the particular compounds, pharmaceutical compositions, uses, and methods described herein, as such matter can, of course, vary. It is also to be understood that the terminology used herein is for the purpose of describing particular embodiments only and is not intended to limit the scope of the present invention, which is only defined herein. As used herein, singular forms of words such as “a,” “an,” and “the” include their corresponding plural referents unless the context clearly dictates otherwise. Thus, e.g., reference to “a compound” includes a single or several different compounds, and reference to “a method” includes reference to equivalent steps and methods known to a person of ordinary skill in the art, and so forth. Unless otherwise defined, all technical and scientific terms used herein have the same meaning as commonly understood by a person of ordinary skill in the art to which this invention belongs.

The techniques that are essential according to the invention are described in detail in the specification. Other techniques which are not described in detail correspond to standard methods that are well known to a person skilled in the art, or the techniques are described in more detail in cited references, patent applications, or standard literature. Although methods and materials similar or equivalent to those described herein can be used in the practice or testing of the present invention, suitable examples are described below. The following examples are provided by way of illustration and not by way of limitation. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again if the technical problem of the invention is solved. Similarly, the features described herein can be combined with one or more other features described herein.

In the following examples, “conventional workup” means: water was added if necessary, the pH was adjusted, if necessary, to a value of between 2 and 10, depending on the constitution of the end product, the mixture was extracted with ethyl acetate (EA) or dichloromethane (DCM), the phases were separated, the organic phase was dried over sodium sulfate and evaporated, and the product was purified by chromatography on silica gel or C-18, and/or by crystallization.

Some abbreviations that may appear in this application are as follows:
ACN acetonitrile
AcOH acetic acid
aq aqueous
API active pharmaceutical ingredient
CDCl₃ deuterated chloroform
CD₃OD deuterated methanol
c-hex cyclohexane
DCC dicyclohexyl carbodiimide
DCM dichloromethane
DIC diisopropyl carbodiimide
DIEA diisopropylethyl-amine
DMF dimethylformamide
DMSO dimethylsulfoxide
DMSO-d6 deuterated dimethylsulfoxide
EDC 1-(3-dimethyl-amino-propyl)-3-ethylcarbodiimide
equiv equivalent
ESI electrospray ionization
Et₂O diethyl ether
EtOAc ethyl acetate
EtOH ethanol
g gram
h hour
HATU dimethylamino-([1,2,3]triazolo[4,5-b]pyridin-3-yloxy)-methylene]-dimethyl-ammonium hexafluorophosphate
HPLC high performance liquid chromatography
i-PrOH 2-propanol
K₂CO₃ potassium carbonate
L liter
LC liquid chromatography
MeOH methanol
mg milligram
MgSO₄ magnesium sulfate
MHz megahertz
min minute
mL milliliter
mm millimeter
mM millimolar
mmol millimole
m.p. melting point
MS mass spectrometry
MTBE methyl tert-butyl ether
NaBH₄ sodium borohydride
NaHCO₃ sodium bicarbonate
NMM N-methyl morpholine
NMR nuclear magnetic resonance
PE petroleum ether
PyBOP benzotriazole-1-yl-oxy-tris-pyrrolidino-phosphonium hexafluorophosphate
Rt retention time
RT room temperature
SPE solid phase extraction
TBTU 2-(1-H-benzotriazole-1-yl)-1,1,3,3-tetramethyl-uromium tetrafluoro borate
TEA triethylamine
TFA trifluoroacetic acid
THF tetrahydrofuran
TLC thin layer chromatography
UV ultraviolet
WL wavelength
μL microliter

### NMR Spectra

¹H NMR was recorded on Bruker DPX-300, DRX-400, AVII-400 or on a 500 MHz spectrometer, using residual signal of deuterated solvent as internal reference. Chemical shifts (δ) were reported in ppm relative to the residual solvent signal (6=2.49 ppm for ¹H NMR in DMSO-d6). ¹H NMR data were reported as follows: chemical shift (multiplicity, coupling constants, and number of hydrogens). Multiplicity was abbreviated as follows: s (singlet), d (doublet), t (triplet), q (quartet), m (multiplet), br (broad).

### LC-MS Methods

| | |
|---|---|
| LC-MS A | Column: Shim-pack XR-ODS, 3.0*50 mm, |
| | 2.2 μm; Mobile Phase A: Water/0.05% TFA, |
| | Mobile Phase B: ACN/0.05% TFA; Flow |
| | rate: 1.2 mL/min; Gradient: 5% B to 100% |
| | B in 2.0 min, hold 0.7 min; 254 nm |
| LC-MS B | Column: Kinetex 1.7 μm C18 100A, |
| | 2.1*30 mm; Column Oven: 40 C.; Mobile |
| | phase A: Water/0.1% FA; Mobile phase B: |
| | Acetonitrile/0.1% FA; Flow rate: 1.0 mL/min; |
| | Gradient: 5% B to 100% B in 2.0 min, hold |
| | 0.6 min; 254 nm |
| LC-MS C | Column: Titank C18, 1.7 μm, 30*2.1 mm; |
| | Column Oven: 40 C.; Mobile Phase A: 0.04% |
| | NH4OH, Mobile Phase B: ACN; Flow rate: |
| | 0.8 mL/min; Gradient: 10% B to 95% B in |
| | 2.1 min, hold 0.6 min; 254 nm |
| LC-MS D | Column: HALO, 3.0*30 mm, 2 μm; Column |
| | Oven: 40 C.; Mobile Phase A: Water/0.0.5% |
| | TFA, Mobile Phase B: ACN/0.05% TFA; |
| | Flow rate: 1.5 mL/min; Gradient: 5% B to |
| | 100% B in 1.2 min, hold 0.5 min; 254 nm |
| LC-MS E | Column: HALO, 3.0*30 mm, 2 μm; Column |
| | Oven: 40 C.; Mobile Phase A: Water/0.05% |
| | TFA, Mobile Phase B: ACN/0.05% TFA; |
| | Flow rate: 1.5 mL/min; Gradient: |
| | 5% B to 95% B in 2.5 min, hold 0.5 min; |
| | 254 nm |
| LC-MS F | Column: ACE Excel 3 SuperC18, 3.0*50 mm, |
| | 3.0 μm; Column Oven: 40 C.; Mobile Phase A: |
| | water/5 mM NH₄HCO₃, Mobile Phase B: |
| | Acetonitrile; Flow rate: 1.2 mL/min; Gradient: |
| | 10% B to 95% B in 2.1 min, hold 0.6 min; |
| | 254 nm |
| LC-MS G | Column: YMC-Triart C18, 3.0 μm, 50*3.0 mm; |
| | Column Oven: 40 C.; Mobile Phase A: 0.04% |
| | NH₄OH, Mobile Phase B: ACN; Flow rate: |
| | 1.2 mL/min; Gradient: 10% B to 95% B in |
| | 2.1 min, hold 0.6 min; 254 nm |
| LC-MS H | Column: XBridge C8, 3.5 μm, 4.6*50 mm; |
| | Solvent A: water + 0.1% TFA; Solvent B: |
| | ACN + 0.1% TFA; Flow: 2 ml/min; Gradient: |
| | 0 min 5% B - 8 min 100% B - 8.1 min: 100% |
| | B, 8.5 min: 5% B, 10 min 5% B |
| LC-MS I | Column: KinetexXB-C18, 1.7 μm, 50*2.1 mm; |
| | Gradient: 0 min 1% B - 0.8 min 99% B - |
| | 1.1 min 99% B, Mobile Phase B: CH₃CN + |
| | 0.1% TFA, Mobile Phase A: H₂O + 0.1% |
| | TFA, Flow: 3.3 ml/min; 254 nm |

### Example 1-A: General Procedures (GP)

### GP A:

To a stirred solution of corresponding arylchloride or arylfluoride (1.00 equiv) and sulfonamide or amine (1.50 equiv) in solvent (39.68 equiv) was added base (2.00 equiv). The resulting mixture was stirred for overnight at 80 degrees C. under nitrogen atmosphere. The resulting mixture was concentrated under vacuum. The residue was purified by silica gel column chromatography to afford desired product.

### GP B:

The corresponding nitrile (1.00 equiv) was dissolved in NH₃ (g) in MeOH (9.67 equiv) and MeOH (52.17 equiv). The resulting mixture was stirred for 16 h at 25 degrees C. under hydrogen atmosphere (balloon). The resulting mixture was filtered, the filter cake was washed with DCM. The filtrate was concentrated under reduced pressure to afford desired product.

### GP C:

To a stirred mixture of arylhalide (1.00 equiv) and amine or sulfonamide (0.90 g, 6.942 mmol, 1.25 equiv, 95%) in solvent (35.00 equiv) was added base (1.50 equiv), ligand (0.10 equiv), Pd source (0.05 equiv). The resulting mixture was stirred for 3 h at 100 degrees C. under nitrogen atmosphere. The mixture was allowed to cool down to room temperature. The resulting mixture was diluted with ethyl acetate (100 mL). The resulting mixture was filtered. The filtrate was concentrated under reduced pressure. The residue was purified by Prep HPLC to afford desired product.

### GP D:

To a stirred solution of corresponding carbonitrile (1.00 equiv) in EtOH (58.83 equiv)/H₂O (126.48 equiv) was added NaOH (2.00 equiv). The resulting mixture was stirred for 2 h at 80 degrees C. The mixture was acidified to pH 5 with HCl (aq.). The resulting mixture was concentrated under reduced pressure. The residue was purified by silica gel column chromatography to afford desired product.

### GP E:

To a stirred solution of corresponding pyrrole (1.00 equiv) in DMSO (106.07 equiv) were added NaH (1.20 equiv) in portions at room temperature. The resulting mixture was stirred for 30 min at room temperature under nitrogen atmosphere. To the above mixture was added corresponding allylchloride (1.50 equiv) at room temperature. The resulting mixture was stirred for additional 2 h at room temperature. The reaction was quenched by the addition of water at 0 degrees C. The resulting mixture was diluted with EA. The resulting mixture was washed with H₂O and brine. dried over anhydrous Na₂SO₄. After filtration, the filtrate was concentrated under reduced pressure. The residue was purified by Prep-TLC to afford desired product.

### GP F:

A round-bottom flask was charged with corresponding nitro compound (1.00 equiv), methanol (99.10 equiv) and Pt/C (0.01 equiv) at room temperature. The resulting mixture was stirred for 2 h at room temperature under hydrogen atmosphere. The resulting mixture was filtered. The filtrate was concentrated under reduced pressure to afford desired product.

### GP G:

To a stirred mixture of corresponding carboxylate (1.00 equiv) in THF (74.79 equiv) was added LiAlH₄ (1.50 equiv) in portions at 0 degrees C. The resulting mixture was stirred for 2 h at room temperature under nitrogen atmosphere. The reaction was quenched by the addition of Na₂SO₄·10H₂O at 0 degrees C. The resulting mixture was filtered, and the filter cake was washed with DCM. The filtrate was concentrated under reduced pressure. The residue was purified by trituration with MeOH. The precipitated solids were collected by filtration to afford desired product.

List of intermediates which can be obtained according to the reference or general procedure (GP) and starting materials (CAS # are commercially available).

| ID | Structure | Starting material |
|---|---|---|
| I.KIO | 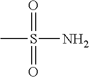 | CAS #: 3144-09-0 |
| I.ZDM | 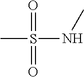 | CAS #: 1184-85-6 |
| I.DES | 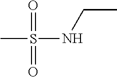 | CAS #: 2374-62-1 |
| I.PXR | 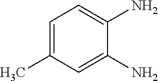 | CAS #: 496-72-0 |
| I.IKB | 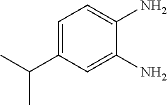 | CAS #: 56471-90-0 |
| I.DMM | 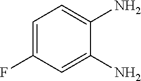 | CAS #: 367-31-7 |
| I.GOL | 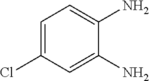 | CAS #: 95-83-0 |
| I.LVP | 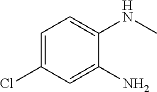 | CAS #: 59681-66-2 |
| I.LEA | 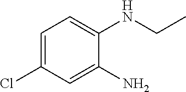 | CAS #: 62476-15-7 |
| I.BXU | 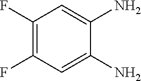 | CAS #: 76179-40-3 |
| I.CUM | 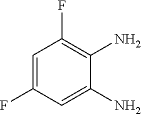 | CAS #: 2369-29-1 |
| I.KIX | 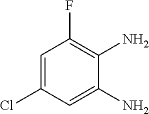 | CAS #: 1106717-48-9 |
| I.KOM | 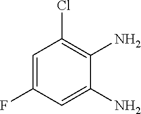 | CAS #: 153505-33-0 |
| I.QZN | 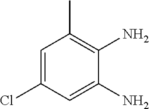 | CAS #: 109671-52-5 |
| I.PET | 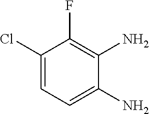 | WO 2015/177367 |
| I.TCK | 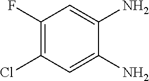 | CAS #: 139512-70-2 |
| I.VWL | 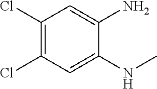 | CAS #: 42450-33-9 |
| I.MCS | 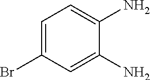 | CAS #: 1575-37-7 |
| I.MIK | 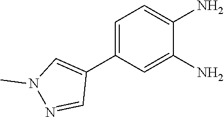 | WO 2014/162039 |
| I.RYP | 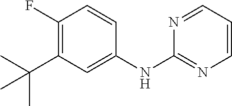 [SMILES: CC(C)(C)c1cc(Nc2ncccn2)ccc1F] | J. Am. Chem. Soc., 2015, vol. 137(43): 13894-13901 |
| I.XVT | 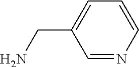 | CAS #: 3731-52-0 |
| I.PJK | 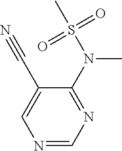 | GP C from I.AJD and I.ZDM with XantPhos, Pd₂(dba)₃, K₃PO₄, toluene, 100° C., 5 h |
| I.QIJ | 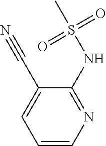 | GP A from I.YGT and I.KIO in DMSO and K₂CO₃, 90° C., 16 h |
| 1.JHQ | 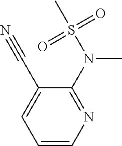 | GP A from I.YGT and I.ZDM in acetone and Cs₂CO₃, 80° C. on |
| I.IIA | 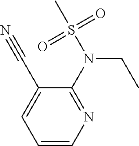 | GP A from I.QIJ and ethyl iodide in DMF and K₂CO₃, 80° C., 16 h |
| I.XDM | 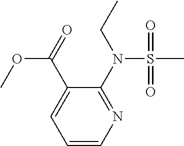 | GP C from I.QND and I.DES with XantPhos, Pd₂(dba)₃, K₃PO₄, toluene, 100° C., 3 h |
| I.PUV | 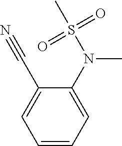 | GP A from I.GJU and I.ZDM in acetonitrile and Cs₂CO₃, 80° C. on |
| I.KOW | 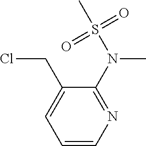 | WO 2015/38417 |
| I.APU | 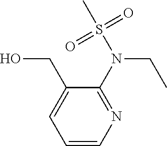 | GP G from I.XDM, 0° C.-25° C., 2 h |
| I.TIJ | 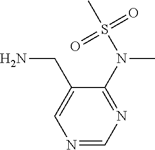 | GP B from I.PJK, 25° C., 2 h |
| I.LWG | 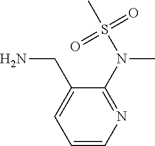 | GP B from I.JHQ |
| I.TIK | 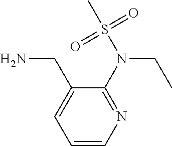 | GP B from I.IIA, 25° C., 16 h |
| I.HAR | 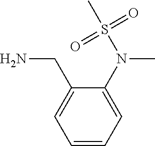 | GP B from I.PUV, 25° C., 16 h |
| I.AJD | 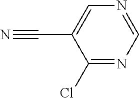 | CAS #: 16357-68-9 |
| I.YGT | 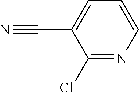 | CAS #: 6602-54-6 |
| I.QND | 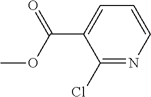 | CAS #: 40134-18-7 |
| I.GJU | 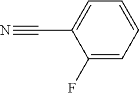 | CAS #: 394-47-8 |
| I.RMQ | 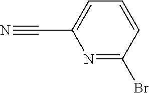 | CAS #: 122918-25-6 |
| I.KAD | 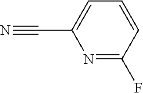 | CAS #: 3939-15-9 |
| I.XOB | 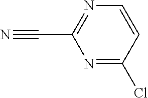 | CAS #: 898044-48-9 |
| I.PEX | 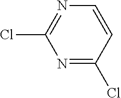 | CAS #: 3934-20-1 |
| I.YLW | 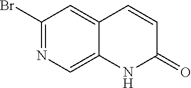 [SMILES: O=c1ccc2cc(Br)ncc2[nH]1] | CAS #: 1574395-48-4 |
| I.VUY | 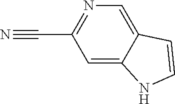 [SMILES: N#Cc1cc2[nH]ccc2cn1] | CAS #: 1082040-98-9 |
| I.YEQ | 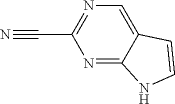 [SMILES: N#Cc1ncc2cc[nH]c2n1] | CAS #: 1211540-09-8 |
| I.POH | 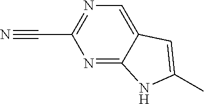 | CAS #: 1638763-44-6 |
| I.OLM | 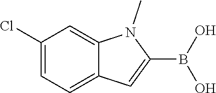 | CAS #: 957066-11-4 |
| I.MUM | 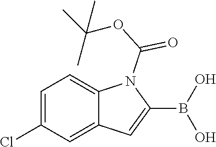 [SMILES: CC(C)(C)OC(=O)n1c(B(O)O)cc2cc(Cl)ccc21] | CAS #: 475102-12-6 |
| I.PPX | 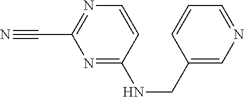 [SMILES: N#Cc1nccc(NCc2cccnc2)n1] | GP A from I.XOB and I.XVT in DCM, 25° C., 1 h |
| I.MPL | 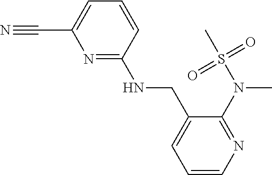 [SMILES: CN(c1ncccc1CNc1cccc(C#N)n1)S(C)(=O)=O] | GP A from I.KAD and I.LWG in NMP and KOtBu, 150° C., 2 h |
| I.WID | 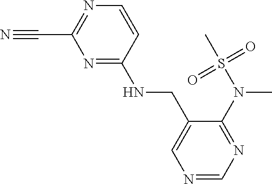 [SMILES: CN(c1ncncc1CNc1ccnc(C#N)n1)S(C)(=O)=O] | GP A from I.XOB and I.TIJ in iPrOH and DIPEA, 80° C., 2 h |
| I.CAL | 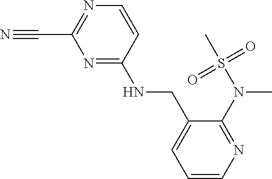 [SMILES: CN(c1ncccc1CNc1ccnc(C#N)n1)S(C)(=O)=O] | GP A from I.XOB and I.LWG in iPrOH and DIPEA, 80° C., 2 h |
| I.UQI | 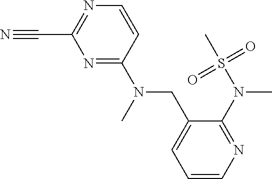 [SMILES: CN(Cc1cccnc1N(C)S(C)(=O)=O)c1ccnc(C#N)n1] | WO 2008/115369 |
| I.FSL | 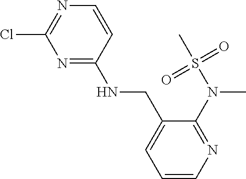 [SMILES: CN(c1ncccc1CNc1ccnc(Cl)n1)S(C)(=O)=O] | GP A from I.PEX and I.LWG in DMA and DIPEA, 60° C., 3 h |
| I.VZZ | 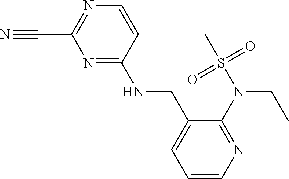 [SMILES: CCN(c1ncccc1CNc1ccnc(C#N)n1)S(C)(=O)=O] | GP A from I.XOB and I.TIK in iPrOH and DIPEA, 80° C., 6 h |
| I.TNF | 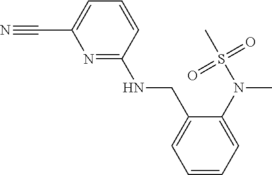 [SMILES: CN(c1ccccc1CNc1cccc(C#N)n1)S(C)(=O)=O] | GP C from I.HAR and I.RMQ with BINAP, Pd₂(dba)₃, NaOtBu, toluene, 120° C., 16 h |
| I.YAN | 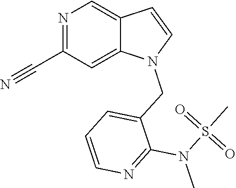 [SMILES: CN(c1ncccc1Cn1ccc2cnc(C#N)cc21)S(C)(=O)=O] | GP E from I.KOW and I.VUY in DMSO and NaH 25° C. 2.5 h |
| I.CHR | 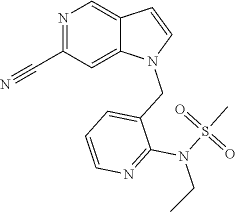 [SMILES: CCN(c1ncccc1Cn1ccc2cnc(C#N)cc21)S(C)(=O)=O] | GP E from I.HOL and I.VUY in DMSO and NaH, 25° C., 2.5 h |
| I.MAR | 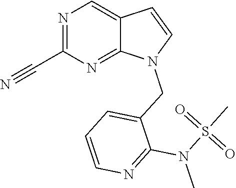 [SMILES: CN(c1ncccc1Cn1ccc2cnc(C#N)nc21)S(C)(=O)=O] | GP E from I.KOW and I.YEQ in DMF and KI (0.20 equiv) and K₂CO₃, 25° C., 2 h |
| I.SAB | 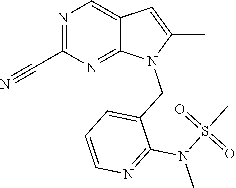 [SMILES: Cc1cc2cnc(C#N)nc2n1Cc1cccnc1N(C)S(C)(=O)=O] | GP E from I.KOW and I.POH in DMSO and NaH, 25° C., 2 h |
| I.HUB | 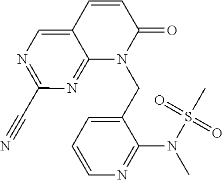 [SMILES: CN(c1ncccc1Cn1c(=O)ccc2cnc(C#N)nc21)S(C)(=O)=O] | GP E from I.KOW and LEYP in DMF and K₂CO₃, 100° C., 2 h |
| I.UHT | 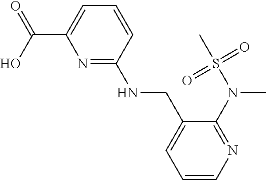 [SMILES: CN(c1ncccc1CNc1cccc(C(=O)O)n1)S(C)(=O)=O] | GP D from I.MPL |
| I.OVA | 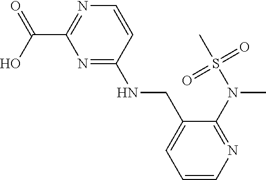 [SMILES: CN(c1ncccc1CNc1ccnc(C(=O)O)n1)S(C)(=O)=O] | GP D from I.CAL |

### Example 1-B: Synthesis of Intermediate N-[3-(chloromethyl)pyridin-2-yl]-N-ethylmethanesulfonamide (I.HOL)

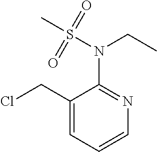

To a stirred solution of N-ethyl-N-[3-(hydroxymethyl)pyridin-2-yl]methanesulfonamide (I.APU, 270 mg, 0.934 mmol, 1.00 equiv, 79.7%) in tetrahydrofuran (25 mL) was added SOCl₂ (5.00 mL) at room temperature. The resulting mixture was stirred for 1 h at 60 degrees C. under nitrogen atmosphere. The resulting mixture was concentrated under vacuum. The resulting mixture was diluted with EA. The residue was basified to pH 9 with saturated Na₂CO₃ (aq.). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by Prep-TLC (PE/EtOAc 2:1) to afford N-[3-(chloromethyl)pyridin-2-yl]-N-ethylmethanesulfonamide (I.HOL, 220 mg, 94.65%) as a white solid.

### Example 1-C: Synthesis of Intermediate 4-tert-butyl-5-fluorobenzene-1,2-diamine (I.HMD)

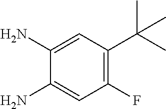

### Step 1: N-(5-tert-butyl-4-fluoro-2-nitrophenyl)pyrimidin-2-amine

To a stirred solution of N-(3-tert-butyl-4-fluorophenyl)pyrimidin-2-amine (I.RYP, 1.00 equiv), silver nitrite (2 equiv), potassium peroxydisulfate (2 equiv) and AcOH (3 equiv) in DCE (97.85 equiv) were added Pd(AcO)₂ (0.1 equiv) at room temperature. The resulting mixture was stirred overnight at 80 degrees C. under 02 atmosphere. The mixture was allowed to cool down to room temperature. The resulting mixture was diluted with H₂O and extracted with EtOAc. The combined organic layers were washed with brine and dried over anhydrous Na₂SO₄. After filtration, the filtrate was concentrated under reduced pressure. The residue was purified by silica gel column chromatography to afford N-(5-tert-butyl-4-fluoro-2-nitrophenyl)pyrimidin-2-amine as a yellow solid.

### Step 2: 5-tert-butyl-4-fluoro-N1-(1,2,3,4-tetrahydropyrimidin-2-yl)benzene-1,2-diamine

To a stirred solution of N-(5-tert-butyl-4-fluoro-2-nitrophenyl)pyrimidin-2-amine (1.00 equiv) in i-PrOH (30 Vequiv) and HCl (2M) (3 equiv) was added Pd/C (0.14 equiv). The resulting mixture was stirred for overnight at 50 degrees C. under hydrogen atmosphere. The resulting mixture was filtered. The filtrate was concentrated under reduced pressure to afford 5-tert-butyl-4-fluoro-N1-(1,2,3,4-tetrahydropyrimidin-2-yl)benzene-1,2-diamine (250 mg, 88.27%) as a light yellow solid.

### Step 3: 4-tert-butyl-5-fluorobenzene-1,2-diamine (I.HMD)

To 5-tert-butyl-4-fluoro-N1-(pyrimidin-2-yl)benzene-1,2-diamine (I.XTV, 1.00 equiv) was added HCl (40.20 equiv). The final reaction mixture was irradiated with microwave radiation for 60 min at 150 degrees C. The mixture was basified to pH 10 with NaOH (30%). The resulting mixture was extracted with EtOEt and dried over anhydrous Na₂SO₄. After filtration, the filtrate was concentrated under reduced pressure. The residue was purified by Prep-TLC to afford 4-tert-butyl-5-fluorobenzene-1,2-diamine (I.HMD, 170 mg, 37.47%) as a brown solid.

### Example 1-D: Intermediate 2-oxo-1,2-dihydro-1,6-naphthyridine-7-carbonitrile (I.EYP)

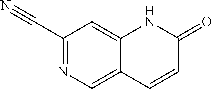 [SMILES: N#Cc1cc2[nH]c(=O)ccc2cn1]

To a stirred solution of 7-bromo-1H-1,6-naphthyridin-2-one (I.YLW, 1.90 g, 7.083 mmol, 1.00 equiv, 83.9%) and Zn(CN)₂ (2.70 g, 21.840 mmol, 3.08 equiv, 95%) in DMF (30.00 mL) was added Pd(PPh3)₄ (1.80 g, 1.402 mmol, 0.20 equiv, 90%). The resulting mixture was stirred for overnight at 115 degrees C. under nitrogen atmosphere. The mixture was allowed to cool down to room temperature. The resulting mixture was diluted with H₂O (30 mL) and extracted with EtOAc (5×30 mL). The combined organic layers were washed with brine (1×30 mL) and dried over anhydrous Na₂SO₄. After filtration, the filtrate was concentrated under reduced pressure. The residue was purified by trituration with EtOAc (10 mL). This resulted in 2-oxo-1,2-dihydro-1,6-naphthyridine-7-carbonitrile (I.EYP, 950 mg, 50.07%) as a light yellow solid.

### Example 1-E: Synthesis of Intermediate 5-chloro-2-(4-chloropyrimidin-2-yl)-1H-indole (I.QXU)

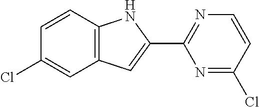 [SMILES: Clc1ccc2[nH]c(-c3nccc(Cl)n3)cc2c1]

### Step 1: tert-butyl 5-chloro-2-(4-methoxypyrimidin-2-yl)-1H-indole-1-carboxylate

To a solution of 1-(tert-butoxycarbonyl)-5-chloroindol-2-ylboronic acid (I.MUM, 1.00 g, 3.215 mmol, 1.00 equiv, 95%) and 2-chloro-4-methoxypyrimidine (CAS #: 22536-63-6, 0.98 g, 6.440 mmol, 2.00 equiv, 95%) in THF (15.00 mL, 208.044 mmol, 57.60 equiv, 100%) and H₂O (1.50 mL, 83.257 mmol, 25.90 equiv, 100%) were added K₂CO₃ (0.94 g, 6.461 mmol, 2.01 equiv, 95%) and Pd(PPh₃)₄ (0.39 g, 0.321 mmol, 0.10 equiv, 95%). After stirring for 16 h at 70 degrees C. under a nitrogen atmosphere, the resulting mixture was concentrated under reduced pressure. The resulting mixture was concentrated under vacuum. The resulting mixture was diluted with water (20 mL). The aqueous layer was extracted with EtOAc (3×20 mL). The resulting mixture was concentrated under vacuum. The residue was purified by Prep-TLC, eluted with PE/EtOAc (5:1) to afford tert-butyl 5-chloro-2-(4-methoxypyrimidin-2-yl)indole-1-carboxylate (1.1 g, 90.35%) as a yellow solid.

### Step 2: 2-(5-chloro-1H-indol-2-yl)pyrimidin-4-ol

To a stirred solution of tert-butyl 5-chloro-2-(4-methoxypyrimidin-2-yl)indole-1-carboxylate (500.00 mg, 1.320 mmol, 1.00 equiv, 95%) in MeCN (10.00 mL, 243.592 mmol, 144.11 equiv, 100%) was added KI (1153.41 mg, 6.601 mmol, 5.00 equiv, 95%), TMSI (1390.27 mg, 6.601 mmol, 5.00 equiv, 95%) in portions at room temperature. The resulting mixture was stirred for 16 h at 80 degrees C. under nitrogen atmosphere. The resulting mixture was diluted with water (15 mL). The aqueous layer was extracted with EtOAc (3×20 mL). The resulting mixture was concentrated under vacuum. The residue was purified by silica gel column chromatography, eluted with PE/EtOAc (1:1) to afford 2-(5-chloro-1H-indol-2-yl)pyrimidin-4-ol (300 mg, 82.23%) as a yellow solid.

### Step 3: 5-chloro-2-(4-chloropyrimidin-2-yl)-1H-indole (I.QXU)

To a stirred solution of 2-(5-chloro-1H-indol-2-yl)pyrimidin-4-ol (I.STV, 200.00 mg, 0.724 mmol, 1.00 equiv, 88.9%) in tetrahydrofuran (25 mL) was added POCl₃ (3.00 mL, 19.565 mmol, 44.47 equiv, 100%) at room temperature. The resulting mixture was stirred for 1 h at 100 degrees C. under nitrogen atmosphere. The reaction was quenched by the addition of sat. NaHCO₃ (aq.) (50 mL) at room temperature. The aqueous layer was extracted with EtOAc (3×10 mL). The resulting mixture was concentrated under vacuum to afford 5-chloro-2-(4-chloropyrimidin-2-yl)-1H-indole (I.QXU, 200 mg, 83.39%) as a yellow solid.

### Example 1-F: Intermediate N-{3-[(5-Bromo-2-cyano-pyrimidin-4-ylamino)-methyl]-pyridin-2-yl}-N-methyl-methanesulfonamide (I.GER)

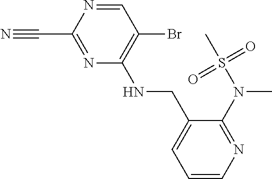 [SMILES: CN(c1ncccc1CNc1nc(C#N)ncc1Br)S(C)(=O)=O]

To a solution of I.CAL (1.00 equiv) in acetonitrile (10.00 Vequiv) was added 1-bromo-pyrrolidine-2,5-dione (1.00 equiv) at −10° C. and the reaction mixture was stirred at room temperature overnight. The reaction mixture was poured into ice water and extracted with ethyl acetate. The organic layer was separated and washed with brine solution, dried over sodium sulphate, and filtered. The filtrate was concentrated under vacuum and purified by silica gel column chromatography to afford product.

### Example 2-A: Synthesis of 2-(5-chloro-1H-benzo[d]imidazol-2-yl)-N-(pyridin-3-ylmethyl)pyrimidin-4-amine

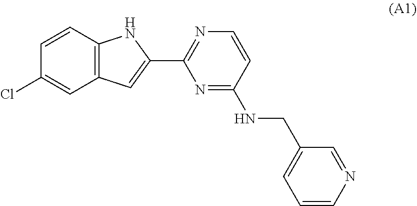 [SMILES: Clc1ccc2[nH]c(-c3nccc(NCc4cccnc4)n3)cc2c1]

To a stirred solution of 4-[(pyridin-3-ylmethyl)amino]pyrimidine-2-carboxylic acid (I.PPX, 1.0 equiv) in POCl₃ (95.51 equiv) was added 4-chloro-1,2-benzenediamine (I.GOL, 1.0 equiv). The resulting mixture was stirred for 16 h at 80 degrees C. under nitrogen atmosphere. The reaction was quenched with sat. aq. NaHCO₃. The resulting mixture was extracted with EtOAc. The combined organic layers were dried over anhydrous Na₂SO₄. After filtration, the filtrate was concentrated under reduced pressure. The residue was purified by silica gel column chromatography to afford the crude product. The crude product was purified by Prep-HPLC.

LC-MS: rt=0.90 min.; Method: LC-MS A

The following compounds were prepared as described for example compound A1 above.

| | | HPLC-MS | Rt | | |
|---|---|---|---|---|---|
| ID | Structure | Method | (min) | NMR | Synthesis |
| A2 | 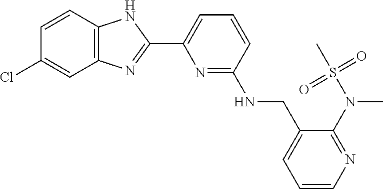 [SMILES: CN(c1ncccc1CNc1cccc(-c2nc3cc(Cl)ccc3[nH]2)n1)S(C)(=O)=O] | LC-MS B | 0.86 | ¹H NMR (300 MHz, DMSO-d6 ppm) δ 12.35- 12.24 (m, 1H), 8.44-8.39 (m, 1H), 7.95-7.89 (m, 1H), 7.73- 7.65 (m, 1H), 7.62-7.52 (m, 2H), 7.50-7.45 (m, 1H), 7.43- 7.37 (m, 1H), | I.UHT and I.GOL, 16 h, 80° C. |
| | | | | 7.34-7.19 (m, | |
| | | | | 2H), 6.72-6.65 | |
| | | | | (m, 1H), 4.91- | |
| | | | | 4.83 (m, 2H), | |
| | | | | 3.26 (s, 6H). | |
| A3 | 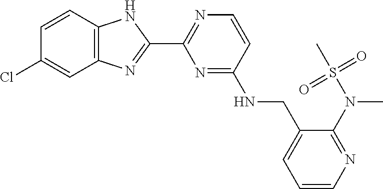 | LC-MS C | 0.99 | ¹H NMR (400 MHz, Methanol- d4 ppm) δ 8.46- 8.40 (m, 1H), 8.31-8.23 (m, 1H), 7.98-7.93 (m, 1H), 7.76- 7.55 (m, 2H), 7.41-7.25 (m, 2H), 6.62 (s, 1H), 5.03 (s, 2H), 3.24 (s, | I.OVA and I.GOL, 6 h, 110° C. |
| | | | | 3H), 3.31 (s, 3H). | |
| A4 | 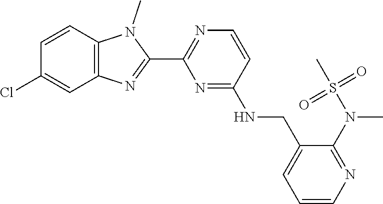 [SMILES: CN(c1ncccc1CNc1ccnc(-c2nc3cc(Cl)ccc3n2C)n1)S(C)(=O)=O] | LC-MS A | 1.23 | ¹H NMR (300 MHz, DMSO, ppm) δ 8.45 (d, J = 4.7 Hz, 1H), 8.36-8.19 (m, 2H), 7.89 (d, J = 7.8 Hz, 1H), 7.77 (s, 1H), 7.67 (d, J = 8.7 Hz, 1H), 7.45 (dd, J = 7.8, 4.8 Hz, 1H), 7.36 (d, J = 8.7 Hz, 1H), 6.72 (s, 1H), | I.OVA and I.LVP. 16 h, 80° C. |
| | | | | 4.89-4.65 (m, | |
| | | | | 2H), 3.95 (s, 3H), | |
| | | | | 3.23 (s, 3H), 3.15 | |
| | | | | (s, 3H). | |

### Example 2-B: Synthesis of N-methyl-N-(3-(((2-(6-methyl-1H-benzo[d]imidazol-2-yl)pyrimidin-4-yl)amino)methyl)pyridin-2-yl)methanesulfonamide

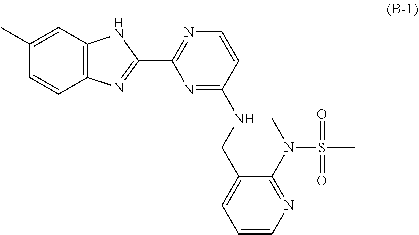

A mixture of 3,4-diaminotoluene (I.PXR, 1.0 equiv) and N-(3-[[(2-cyanopyrimidin-4-yl)amino]methyl]pyridin-2-yl)-N-methylmethanesulfonamide (I.CAL, 1 equiv) was stirred for 2 h at 160 degrees C. under argon atmosphere. The residue was purified by reverse flash chromatography. The crude product was purified by Prep-HPLC to afford desired product.

The following compounds were prepared as described for example compound B1 above.

| | | HPLC-MS | Rt | | |
|---|---|---|---|---|---|
| ID | Structure | Method | (min) | NMR | Synthesis |
| B1 | 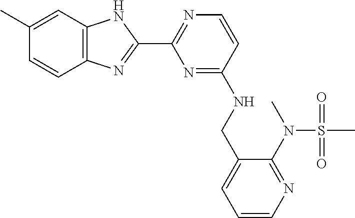 | LC-MS A | 1.15 | ¹H NMR (300 MHz, Methanol- d4) δ 8.42 (d, J = 4.8 Hz, 1H), 8.24 (d, J = 6.0 Hz, 1H), 7.93 (d, J = 7.8 Hz, 1H), 7.53 (d, J = 8.3 Hz, 1H), 7.43 (s, 1H), 7.41-7.31 (m, 1H), 7.14 (d, J = 8.4 Hz, 1H), 6.58 (s, 1H), | I.PXR and I.CAL, 2 h, 160° C., Ar |
| | | | | 5.01 (s, 2H), | |
| | | | | 3.33 (d, J = 1.6 | |
| | | | | Hz, 3H), 3.23 (s, | |
| | | | | 3H), 2.47 (s, | |
| | | | | 3H). | |
| B2 | 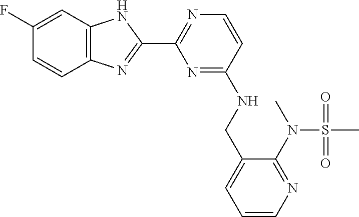 | LC-MS I | 0.77 | ¹H NMR (500 MHz, DMSO) δ 12.84 (s, 1H), 8.44 (dd, J = 4.9, 1.9 Hz, 1H), 8.26 (s, 1H), 8.15 (d, J = 9.2 Hz, 1H), 7.96 (s, 1H), 7.55-7.44 (m, 1H), 7.42 (dd, J = 7.8, 4.7 Hz, 1H), 7.29- 7.04 (m, 1H), | I.DMM and I.CAL, 2 h, 160° C., Ar |
| | | | | 6.64 (s, 1H), | |
| | | | | 4.84 (s, 2H), | |
| | | | | 3.18 (s, 3H). | |
| B3 | 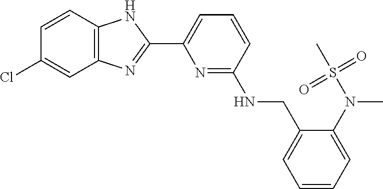 | LC-MS D | 1.26 | ¹H NMR (300 MHz, DMSO-d6 ppm) 12.35- 12.24 (m, 1H), 8.44-8.39 (m, 1H), 7.95-7.89 (m, 1H), 7.73- 7.65 (m, 1H), 7.62-7.52 (m, 2H), 7.50-7.45 (m, 1H), 7.43- 7.37 (m, 1H), | I.GOL and I.TNF, 3 h, 160° C., Ar |
| | | | | 7.34-7.19 (m, | |
| | | | | 2H), 6.72-6.65 | |
| | | | | (m, 1H), 4.91- | |
| | | | | 4.83 (m, 2H), | |
| | | | | 3.26 (s, 6H). | |
| B4 | 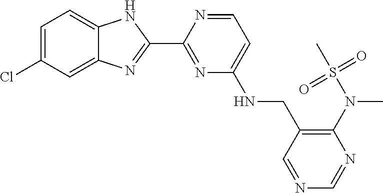 | LC-MS A | 1.13 | ¹H NMR (400 MHz, DMSO-d6) δ 12.99 (d, J = 24.9 Hz, 1H), 9.07 (s, 1H), 9.01 (s, 1H), 8.31-8.21 (m, 2H), 7.78-7.68 (m, 1H), 7.57- 7.51 (m, 1H), 7.26 (ddd, J = 21.4, 8.6, 2.1 | I.GOL and I.WID, 3 h, 160° C., Ar |
| | | | | Hz, 1H), 6.65 (s, | |
| | | | | 1H), 4.79 (s, | |
| | | | | 2H), 3.47-3.35 | |
| | | | | (m, 3H), 3.26 (d, | |
| | | | | J = 1.5 Hz, 3H). | |
| B5 | 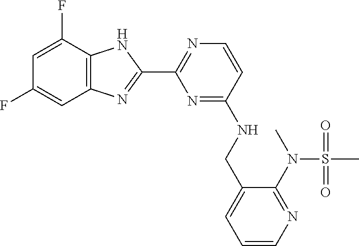 | LC-MS A | 1.18 | ¹H NMR (300 MHz, Methanol- d4) δ 8.44 (s, 1H), 8.26 (d, J = 5.9 Hz, 1H), 7.94 (d, J = 7.7 Hz, 1H), 7.38 (dd, J = 7.6, 4.7 Hz, 1H), 7.17 (d, J = 8.4 Hz, 1H), 6.92 (t, J = 10.4 Hz, 1H), 6.63 (s, 1H), 5.04 (s, 2H), 3.32 (d, J = 1.7 Hz, 3H), | I.CUM and I.CAL, 2 h, 160° C., Ar |
| | | | | 3.23 (s, 3H). | |
| B6 | 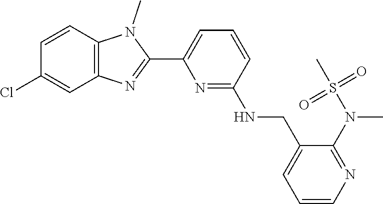 | LC-MS A | 1.22 | ¹H NMR (400 MHz, DMSO-d6) δ 8.42 (dd, J = 4.8, 1.9 Hz, 1H), 7.82 (dd, J = 7.7, 1.9 Hz, 1H), 7.71 (d, J = 2.0 Hz, 1H), 7.69- 7.52 (m, 2H), 7.50-7.36 (m, 3H), 7.28 (dd, J = 8.7.2.0 Hz, 1H), 6.74 (d, J = | L.LVP and I.MLP, 2 h, 160° C., Ar |
| | | | | 8.3 Hz, 1H), | |
| | | | | 4.74 (d, J = 5.8 | |
| | | | | Hz, 2H), 3.91 (s, | |
| | | | | 3H), 3.17 (d, J = | |
| | | | | 10.3 Hz, 6H). | |
| B7 | 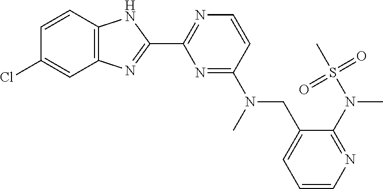 | LC-MS A | 1.21 | ¹H NMR (400 MHz, DMSO-d6) δ 12.98 (s, 1H), 8.46 (dd, J = 4.7, 1.8 Hz, 1H), 8.38 (s, 1H), 7.80-7.69 (m, 2H), 7.55 (d, J = 8.3 Hz, 1H), 7.40 (dd, J = 7.7, 4.7 Hz, 1H), 7.26 (dd, J = | I.GOL and I.UQI, 3 h, 160° C., Ar |
| | | | | 20.8, 8.6 Hz, | |
| | | | | 1H), 6.73 (s, | |
| | | | | 1H), 5.09 (s, | |
| | | | | 2H), 3.32 (s, | |
| | | | | 3H), 3.28 (s, | |
| | | | | 3H), 3.19 (s, | |
| | | | | 3H). | |
| B8 | 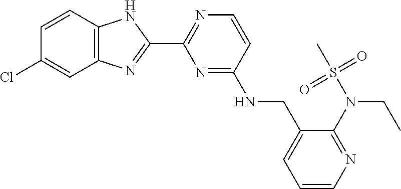 | LC-MS D | 1.17 | ¹H NMR (400 MHz, DMSO-d6) δ 12.91 (s, 1H), 8.47 (d, J = 3.9 Hz, 1H), 8.27 (d, J = 6.0 Hz, 1H), 8.12 (s, 1H), 7.94 (s, 1H), 7.77 (m, 1H), 7.53 (s, 1H), 7.43 (dd, J = 7.7, 4.7 Hz, 1H), | I.GOL and I.VZZ, On, 160° C., Ar |
| | | | | 7.28 (dd, J = | |
| | | | | 23.1, 14.8 Hz, | |
| | | | | 1H), 6.70 (s, | |
| | | | | 1H), 4.86 (s, | |
| | | | | 2H), 3.77 (q, J = | |
| | | | | 7.1 Hz, 2H), | |
| | | | | 3.14 (s, 3H), | |
| | | | | 1.09 (t, J = 7.2 | |
| | | | | Hz, 3H). | |
| B9 | 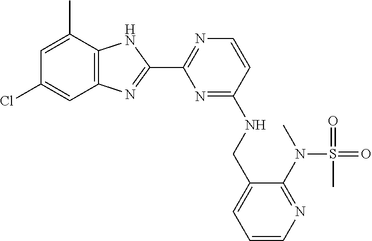 | LC-MS E | 1.26 | ¹H NMR (400 MHz, Methanol- d4) δ 8.44 (d, J = 4.5 Hz, 1H), 8.27 (d, J = 6.0 Hz, 1H), 7.94 (d, J = 7.9 Hz, 1H), 7.39 (dd, J = 7.8, 4.7 Hz, 2H), 7.10 (s, 1H), 6.63 (s, 1H), 5.06 (s, 2H), 3.32 (s, 3H), 3.23 (s, 3H), 2.64 (s, 3H). | I.QZN and I.CAL, 2 h 160° C., Ar |
| B10 | 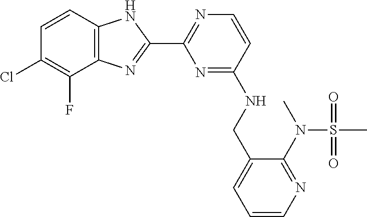 | LC-MS A | 1.23 | ¹H NMR (400 MHz, DMSO-d6) δ 13.29 (s, 1H), 8.44 (d, J = 4.6 Hz, 1H), 8.28 (s, 2H), 7.95 (s, 1H), 7.46-7.40 (m, 1H), 7.43- 7.34 (m, 2H), 6.68 (s, 1H), 4.83 (s, 2H), 3.39 (s, 3H), 3.18 (s, 3H). | I.PET and I.CAL, 2 h 160° C., Ar |
| B11 | 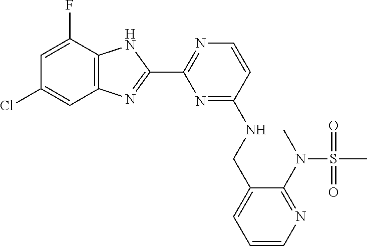 | LC-MS A | 1.26 | ¹H NMR (300 MHz, DMSO-d6) δ 13.28 (s, 1H), 8.49-8.40 (m, 1H), 8.29 (s, 2H), 7.96 (s, 1H), 7.51-7.29 (m, 2H), 7.22 (d, J = 10.4 Hz, 1H), 6.69 (s, 1H), 4.83 (s, 2H), 3.39 (s, 3H), 3.18 (s, 3H) | I.KIX and I.CAL, 2 h, 160° C., Ar |
| B12 | 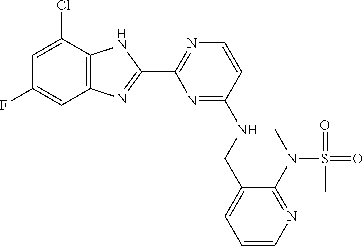 | LC-MS A | 1.23 | ¹H NMR (300 MHz, DMSO-d6) δ 13.24 (s, 1H), 8.36 (dd, J = 46.7, 5.2 Hz, 3H), 7.97 (s, 1H), 7.44 (dd, J = 7.9, 4.8 Hz, 1H), 7.31 (t, J = 9.6 Hz, 2H), 6.70 (s, 1H), 4.83 (s, 2H), 3.46 (s, 3H), 3.20 (d, J = 1.2 Hz, 3H). | I.KOM and I.CAL, 2 h 160° C., Ar |
| B13 | 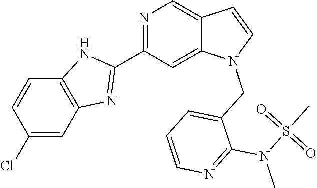 | LC-MS D | 1.11 | ¹H NMR (400 MHz, DMSO-d6) δ 13.06 (s, 1H), 9.04 (d, J = 1.0 Hz, 1H), 8.48 (dd, J = 4.7, 1.8 Hz, 1H), 8.32 (dd, J = 3.9, 1.1 Hz, 1H), 7.70 (d, J = 3.2 Hz, 2H), 7.68 (m, 1H), 7.54 (m, 1H), | I.GOL and I.YAN, On, 160° C., Ar |
| | | | | 7.38 (dd, J = | |
| | | | | 7.8, 4.7 Hz, 2H), | |
| | | | | 6.84 (dd, J = | |
| | | | | 3.2, 0.9 Hz, 1H), | |
| | | | | 5.75 (s, 2H), | |
| | | | | 3.26 (m, 3H), | |
| | | | | 3.19 (m, 3H). | |
| B14 | 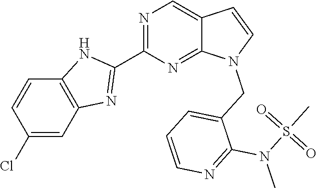 | LC-MS D | 1.10 | ¹H NMR (400 MHz, DMSO-d6) δ 13.22 (s, 1H), 9.24 (s, 1H), 8.49 (dd, J = 4.7, 1.9 Hz, 1H), 7.79 (d, J = 3.5 Hz, 2H), 7.75 (m, 1H), 7.57 (s, 1H), 7.45 (dd, J = 7.8, 1.9 Hz, 1H), 7.37 (dd, | I.GOL and I.MAR, On, 160° C., Ar |
| | | | | J = 7.8, 4.7 Hz, | |
| | | | | 1H), 7.27 (s, | |
| | | | | 1H), 5.76 (s, | |
| | | | | 2H), 3.36 (s, | |
| | | | | 3H), 3.20 (s, | |
| | | | | 3H). | |
| B15 | 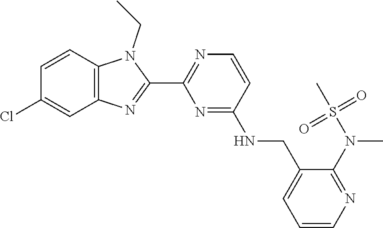 | LC-MS A | 1.27 | ¹H NMR (400 MHz, DMSO-d6) δ 8.47-8.41 (m, 1H), 8.30 (d, J = 6.0 Hz, 1H), 8.24 (s, 1H), 7.87 (d, J = 7.7 Hz, 1H), 7.75 (d, J = 2.0 Hz, 1H), 7.68 (d, J = 8.7 Hz, 1H), 7.44 (dd, J = 7.7, 4.7 Hz, 1H), 7.33 (dd, J = 8.6, 2.0 | I.LEA and I.CAL, 3 h, 160° C., Ar |
| | | | | Hz, 1H), 6.70 (s, | |
| | | | | 1H), 4.73 (s, | |
| | | | | 2H), 4.53 (s, | |
| | | | | 2H), 3.20 (s, | |
| | | | | 3H), 3.13 (s, | |
| | | | | 3H), 1.16 (s, | |
| | | | | 3H | |
| B16 | 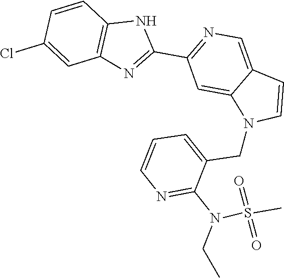 | LC-MS A | 1.27 | ¹H NMR (400 MHz, DMSO-d6) δ 13.08 (s, 1H), 9.05 (d, J = 1.0 Hz, 1H), 8.52 (dd, J = 4.7, 1.8 Hz, 1H), 8.28 (s, 1H), 7.69 (d, J = 3.2 Hz, 1H), 7.66-7.57 (m, 1H), 7.50 (s, 1H), 7.37 (dd, J = 7.8, 4.7 Hz, 1H), 7.20 (s, 1H), 7.04 (d, J = 7.3 Hz, 1H), 6.86 (dd, J = | I.GOL and I.CHR, 2 h, 160° C., Ar |
| | | | | 3.3, 0.9 Hz, 1H), | |
| | | | | 5.76 (s, 2H), | |
| | | | | 3.79 (q, J = 7.1 | |
| | | | | Hz, 2H), 3.17 (s, | |
| | | | | 3H), 1.06 (t, J = | |
| | | | | 7.2 Hz, 3H). | |
| B17 | 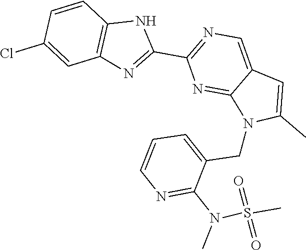 | LC-MS A | 1.29 | ¹H NMR (300 MHz, DMSO-d6) δ 13.24 (s, 1H), 9.14 (s, 1H), 8.50 (d, J = 4.7 Hz, 1H), 7.80- 7.68 (m, 1H), 7.57 (d, J = 6.6 Hz, 1H), 7.39- 7.20 (m, 2H), 7.06 (d, J = 7.9 Hz, 1H), 6.63 (s, 1H), 5.76 (s, 2H), 3.40 (s, 3H), 3.23 (s, | I.GOL and I.SAB, 2 h, 160° C., Ar |
| | | | | 3H), 2.36 (s, | |
| | | | | 3H). | |
| B18 | 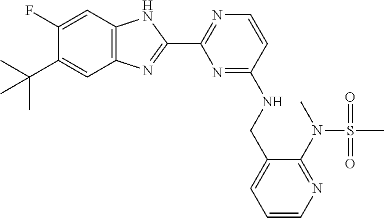 | LC-MS A | 1.42 | ¹H NMR (300 MHz, Methanol- d4) δ 8.44 (s, 1H), 8.26 (d, J = 6.1 Hz, 1H), 7.96 (d, J = 7.7 Hz, 1H), 7.62 (s, 1H), 7.43-7.34 (m, 1H), 7.30 (d, J = 12.6 Hz, 1H), 6.61 (s, 1H), 5.03 (s, 2H), 3.32 (d, J = | I.HMD and I.CAL, 2 h, 160° C., Ar |
| | | | | 1.5 Hz, 3H), | |
| | | | | 3.24 (s, 3H), | |
| | | | | 1.47 (s, 9H). | |
| B19 | 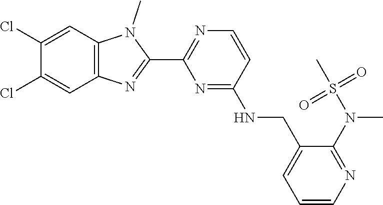 | LC-MS D | 1.24 | ¹H NMR (300 MHz, DMSO-d6) δ 8.48 (m, 1H), 8.31 (d, J = 6.1 Hz, 2H), 8.05 (s, 1H), 7.98 (s, 1H), 7.87 (d, J = 7.8 Hz, 1H), 7.44 (dd, J = 7.8, 4.7 Hz, 1H), 6.72 (s, 1H), 4.75 (s, 2H), 3.93 (s, 3H), | I.VWL and I.CAL, On, 160° C., Ar |
| | | | | 3.21 (s, 3H), | |
| | | | | 3.14 (s, 3H). | |
| B20 | 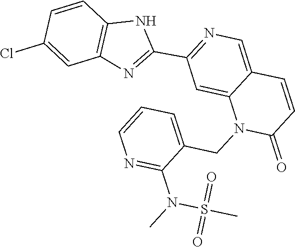 | LC-MS A | 1.45 | ¹H NMR (300 MHz, DMSO-d6) δ 13.34 (d, J = 21.9 Hz, 1H), 9.06 (s, 1H), 8.46 (dd, J = 4.5, 1.8 Hz, 1H), 8.18 (d, J = 9.5 Hz, 1H), 7.87 (s, 1H), 7.70-7.57 (m, 1H), 7.55- 7.46 (m, 1H), 7.38 (d, J = 7.5 Hz, 1H), 7.35- 7.17 (m, 2H), | I.GOL and I.HUB, 2 h, 160° C., Ar |
| | | | | 6.89 (dd, J = | |
| | | | | 9.5, 0.9 Hz, 1H), | |
| | | | | 5.58 (s, 2H), | |
| | | | | 3.67 (d, J = 2.9 | |
| | | | | Hz, 3H), 3.15 (s, | |
| | | | | 3H). | |
| B21 | 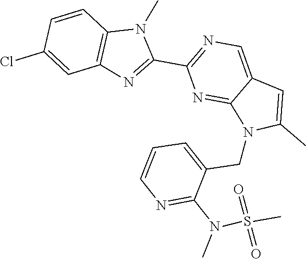 | LC-MS A | 1.31 | ¹H NMR (300 MHz, DMSO-d6) δ 9.14 (s, 1H), 8.46 (d, J = 4.9 Hz, 1H), 7.76 (s, 1H), 7.69 (d, J = 8.7 Hz, 1H), 7.34 (ddd, J = 9.1, 7.6, 3.4 Hz, 2H), 7.00 (d, J = 7.9 Hz, 1H), 6.68-6.61 (m, 1H), 5.71 (s, 2H), 4.10 (s, 3H), 3.28 (s, 3H), 3.17 (s, | I.LVP and I.SAB, 2 h, 160° C., Ar |
| | | | | 3H), 2.38 (s, | |
| | | | | 3H). | |
| B22 | 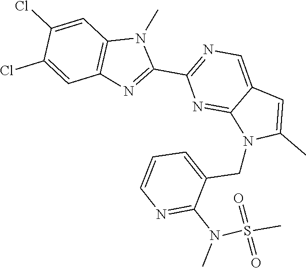 | LC-MS A | 1.45 | ¹H NMR (300 MHz, DMSO-d6) δ 9.15 (s, 1H), 8.46 (dd, J = 4.8, 1.7 Hz, 1H), 8.05 (s, 1H), 7.98 (s, 1H), 7.32 (dd, J = 7.8, 4.8 Hz, 1H), 6.99 (d, J = 7.0 Hz, 1H), 6.65 (d, J = 1.2 Hz, 1H), 5.70 (s, 2H), 4.09 (s, 3H), 3.28 (s, 3H), 3.17 (s, 3H), | I.VWL and I.SAB, 1 h, 160° C., Ar |
| | | | | 2.38 (d, J = 1.0 | |
| | | | | Hz, 3H), 1.24 (s, | |
| | | | | 1H). | |
| B23 | 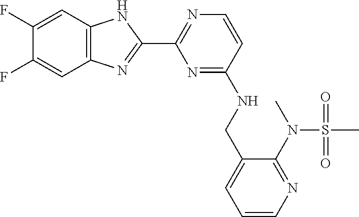 | LC-MS I | 0.78 | ¹H NMR (500 MHz, DMSO) δ 12.97 (s, 1H), 8.44 (dd, J = 4.9, 1.8 Hz, 1H), 8.25 (d, J = 5.4 Hz, 1H), 8.18 (s, 1H), 7.95 (s, 1H), 7.87-7.45 (m, 3H), 7.42 (dd, J = 7.7, 4.7 Hz, 1H), 6.65 (s, 1H), 4.82 (s, | I.BXU and I.CAL, 2 h, 160° C., Ar |
| | | | | 2H), 3.18 (s, | |
| | | | | 3H). | |
| B24 | 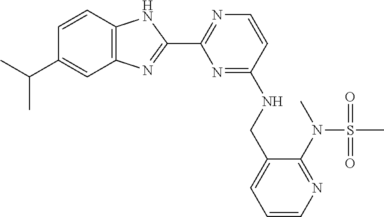 | LC-MS I | 0.83 | ¹H NMR (500 MHz, DMSO) δ 12.89 (s, 1H), 8.44 (dd, J = 4.9, 1.8 Hz, 1H), 8.28-8.17 (m, 2H), 7.97 (s, 1H), 7.54 (d, J = 8.4 Hz, 1H), 7.45-7.39 (m, 2H), 7.17 (dd, J = 8.5, 1.6 Hz, 1H), 6.64 (s, | I.IKB and I.CAL, 2 h, 160° C., Ar |
| | | | | 1H), 4.86 (s, | |
| | | | | 2H), 3.18 (s, | |
| | | | | 3H), 3.02 (p, J = | |
| | | | | 6.9 Hz, 1H), | |
| | | | | 1.26 (d, J = 6.9 | |
| | | | | Hz, 6H). | |
| B25 | 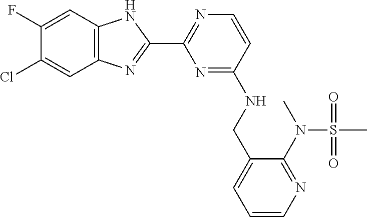 | LC-MS I | 0.809 | ¹H NMR (500 MHz, DMSO) δ 8.83 (s, 1H), 8.47 (dd, J = 4.7, 1.9 Hz, 1H), 8.24 (s, 1H), 7.99 (s, 1H), 7.88 (d, J = 6.7 Hz, 1H), 7.71- 7.65 (m, 1H), 7.44 (dd, J = 7.7, 4.7 Hz, 1H), 6.73 (s, 1H), | I.TCK and I.CAL, 2 h 160° C., Ar |
| | | | | 4.95 (s, 2H), | |
| | | | | 3.28 (s, 3H), | |
| | | | | 3.17 (s, 3H). | |
| B26 | 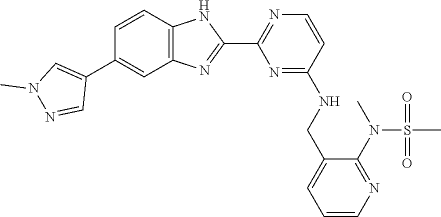 | LC-MS I | 0.77 | ¹H NMR (500 MHz, DMSO) δ 12.69 (s, 1H), 8.43 (dd, J = 4.8, 1.9 Hz, 1H), 8.25 (d, J = 5.7 Hz, 1H), 8.17- 8.10 (m, 2H), 7.96 (s, 1H), 7.84 (s, 2H), 7.61 (s, 2H), 7.42 (dd, J = 7.8, 4.7 Hz, 1H), | I.MIK and I.CAL, 2 h, 160° C., Ar |
| | | | | 6.63 (s, 1H), | |
| | | | | 4.84 (s, 2H), | |
| | | | | 3.88 (s, 3H), | |
| | | | | 3.35 (s, 3H), | |
| | | | | 3.19 (s, 3H). | |

### Example 2-E: Synthesis of N-(3-{[5-Bromo-2-(5-chloro-1H-benzoimidazol-2-yl)-pyrimidin-4-ylamino]-methyl}-pyridin-2-yl)-N-methyl-methanesulfonamide (E1)

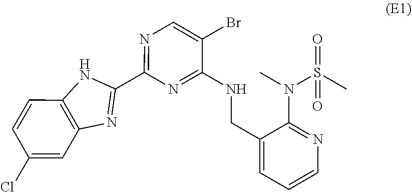 [SMILES: CN(c1ncccc1CNc1nc(-c2nc3cc(Cl)ccc3[nH]2)ncc1Br)S(C)(=O)=O]

To a solution of I.GER (1.00 equiv) in methanol (20.00 Vequiv) was added sodium methoxide (25% w/v in methanol) (2.00 equiv) and I.GOL (1.00 equiv) at room temperature and stirred the reaction mass for 3 h at 60 degrees C. After the completion of the reaction as evidenced by TLC, the reaction mixture was evaporated under vacuum and quenched with H₂O and extracted with ethyl acetate. The organic layer was separated and dried using Na₂SO₄ and filtered. The filtrate was concentrated under vacuum and the crude reaction mixture was purified using silica gel column chromatography to afford N-(3-{[5-Bromo-2-(5-chloro-1H-benzoimidazol-2-yl)-pyrimidin-4-ylamino]-methyl}-pyridin-2-yl)-N-methyl-methanesulfonamide as white yellow powder.

LC-MS: rt: 3.47 min; Method: LC-MS H.

¹H NMR: 400 MHz, DMSO-d6: 13.00-12.39 (m, 1H), 9.52 (s, 1H), 8.42-8.410 (m, 1H), 8.32-8.26 (m, J=6.00 Hz, 1H), 7.87-7.85 (m, 1H), 6.51-6.49 (d, J=8.68 Hz, 1H), 7.23 (s, 1H), 7.40-7.37 (m, 1H), 7.29-7.26 (m, 1H), 4.90-4.88 (m, 2H), 3.43 (s, 3H), 3.16 (s, 3H).

### Example 3: Biochemical Assays for Assessing SRPK1, SRPK2 and SRPK3 Inhibition

### Buffer Conditions

20 mM HEPES (pH 7.5), 10 mM MgCl₂, 1 mM EGTA, 0.01% Brij35, 0.02 mg/ml BSA, 0.1 mM Na₃VO₄, 2 mM DTT, 1% DMSO

### Reaction Procedure

The indicated substrate was prepared in freshly prepared reaction buffer. The required cofactors were added individually to the substrate solution above. The indicated kinase was delivered into the substrate solution and mixed gently. The compounds in DMSO were delivered into the kinase reaction mixture utilizing acoustic technology (Echo550). ³³P-ATP (specific activity 0.01 μCi/μl final) was delivered into the reaction mixture to initiate the reaction. The kinase reaction was incubated for 120 minutes at room temperature. Reactions were spotted onto P81 ion exchange paper (Whatman #3698-915). The filters were extensively washed in 0.75% phosphoric acid. The radioactive phosphorylated substrate remaining on the filter paper was measured.

### Data Analysis

Kinase activity data were expressed as the percent remaining kinase activity in test samples compared to vehicle (dimethyl sulfoxide) reactions. To pass QC, all DMSO control values must have a coefficient of variation less than 10% and the internal control IC₅₀ value be within 3-fold of the 6 months historical average. IC₅₀ values and curve fits were obtained using Prism4 Software (GraphPad). The equation used for curve fitting was:

$$Y = Bottom + ( Top - Bottom ) / ( 1 + 10 \hat{} ( {( Log {IC}_{50} - X )}^{*} HillSlope ) ) .$$

### Substrate and Co-Factor Information

| | | Substrates in |
|---|---|---|
| Kinases | Substrate | Reaction (μM) |
| SRPK1 | RS Peptide | 20 |
| SRPK2 | RS Peptide | 20 |
| MSSK1/STK23 | RS Peptide | 20 |

The compound inhibition (IC₅₀) is given below:

| | | | SRPK3/ |
|---|---|---|---|
| Compound | SRPK1 | SRPK2 | MSSK1/STK23 |
| A2 | A | B | |
| A3 | A | B | A |
| A4 | B | B | |
| B1 | A | C | A |
| B2 | B | C | A |
| B3 | A | B | |
| B4 | B | C | |
| B5 | A | C | A |
| B6 | B | B | A |
| B7 | A | C | |
| B8 | A | B | |
| B9 | A | B | A |
| B10 | A | B | A |
| B11 | A | B | A |
| B12 | A | C | A |
| B13 | B | B | B |
| B14 | A | B | |
| B15 | A | C | |
| B16 | B | B | B |
| B17 | A | B | B |
| B18 | B | C | A |
| B19 | | B | |
| B20 | B | B | C |
| B21 | A | B | B |
| B22 | A | B | B |
| B23 | A | C | A |
| B24 | A | C | A |
| B25 | A | C | A |
| B26 | A | C | B |
| E1 | A | C | B |

(A < 50 nM, 50 ≤ B < 500 nM, and 500 ≤ C < 2500 nM)

### Example 4: Pharmaceutical Preparations

(A) Injection vials: A solution of 133 g of an API according to the invention and 5 g of disodium hydrogen phosphate in 3 l of bidistilled water was adjusted to pH 6.5 using 2 IN hydrochloric acid, sterile filtered, transferred into injection vials, lyophilized Linder sterile conditions and sealed under sterile conditions. Each injection vial contained 5 mg of API.
(B) Suppositories: A mixture of 20 g of an API according to the invention was melted with 100 g of soy lecithin and 1400 g of cocoa butter, poured into molds, and allowed to cool. Each suppository contained 20 mg of API.
(C) Solution: A solution was prepared from 1 g of an API according to the invention, 9.38 g of NaH₂PO₄·2H₂O, 28.48 g of Na₂HPO₄·12H₂O and 0.1 g of benzalkonium chloride in 940 ml of bidistilled water. The pH was adjusted to 6.8, and the solution was made up to 1 l and sterilized by irradiation. This solution could be used in the form of eye drops.
(D) Ointment: 500 mg of an API according to the invention were mixed with 99.5 g of Vaseline under aseptic conditions.
(E) Tablets: A mixture of 1 kg of an API according to the invention, 4 kg of lactose, 1.2 kg of potato starch, 0.2 kg of talc and 0.1 kg of magnesium stearate was pressed to give tablets in a conventional manner in such a way that each tablet contained 10 mg of API.
(F) Coated tablets: Tablets were pressed analogously to Example E and subsequently coated in a conventional manner with a coating of sucrose, potato starch, talc, tragacanth, and dye.
(G) Capsules: 2 kg of an API according to the invention were introduced into hard gelatin capsules in a conventional manner in such a way that each capsule contained 20 mg of API.
(H) Ampoules: A solution of 1 kg of an API according to the invention in 60 l of bidistilled water was sterile filtered, transferred into ampoules, lyophilized under sterile conditions, and sealed under sterile conditions. Each ampoule contained 10 mg of active ingredient.
(I) Inhalation spray: 14 g of an API according to the invention were dissolved in 10 l of isotonic NaCl solution, and the solution was transferred into commercially available spray containers with a pump mechanism. The solution could be sprayed into the mouth or nose. One spray shot (about 0.1 ml) corresponded to a dose of about 0.14 mg.

1. A compound of formula (I), 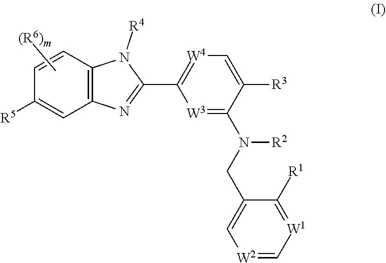
wherein
W¹, W², W³, W⁴ denote independently from one another N or CH;
R¹ denotes NYSO₂Y or Y;
R², R⁴ denote Y;
R³ denotes Y or Hal;
R², R³ together also denote —(CY)₂- or —(CRI)—(CY)₂-;
R⁵, R⁶ denote independently from one another Hal, Y or Het;
R⁷ denotes Y or ═O;
Y denotes H or A;
A denotes unbranched or branched alkyl having 1-10 C atoms, in which 1-7 H atoms can be replaced independently from one another by Hal;
Het denotes an optionally substituted, saturated, unsaturated or aromatic monocyclic 5-6-membered heterocycle having 2-5 C atoms and 1-3 N, O and/or S atoms;
Hal denotes F, Cl, Br or I; and
m denotes 0, 1, 2 or 3;
and/or a physiologically acceptable salt thereof;
with the proviso that 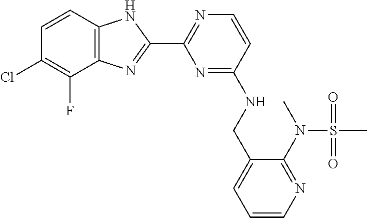 [SMILES: CN(c1ncccc1CNc1ccnc(-c2nc3c(F)c(Cl)ccc3[nH]2)n1)S(C)(=O)=O]
is excluded.

2. The compound according to claim 1, wherein
W² denotes CH, and
W¹, W³ and/or W⁴ denote N.

3. The compound according to claim 1, wherein
R¹ denotes NASO₂A.

4. The compound according to claim 1, wherein
R², R³ and/or R⁴ denote H, or
R², R³ together denote —(CY)₂- with the proviso that W⁴ denotes N.

5. The compound according to claim 1, wherein
R⁵, R⁶ denote independently from one another Hal or A, and/or
m denotes 0 or 1.

6. The compound according to claim 1, having sub-formula (I-A) 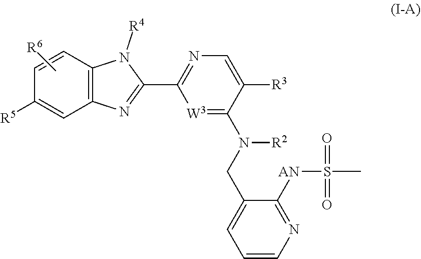
wherein
R⁵, R⁶ denote independently from one another Hal or A, and
W³, R², R³, R⁴ and A have the meaning as defined in claim 1.

7. The compound according to claim 1, which is selected from the group consisting of 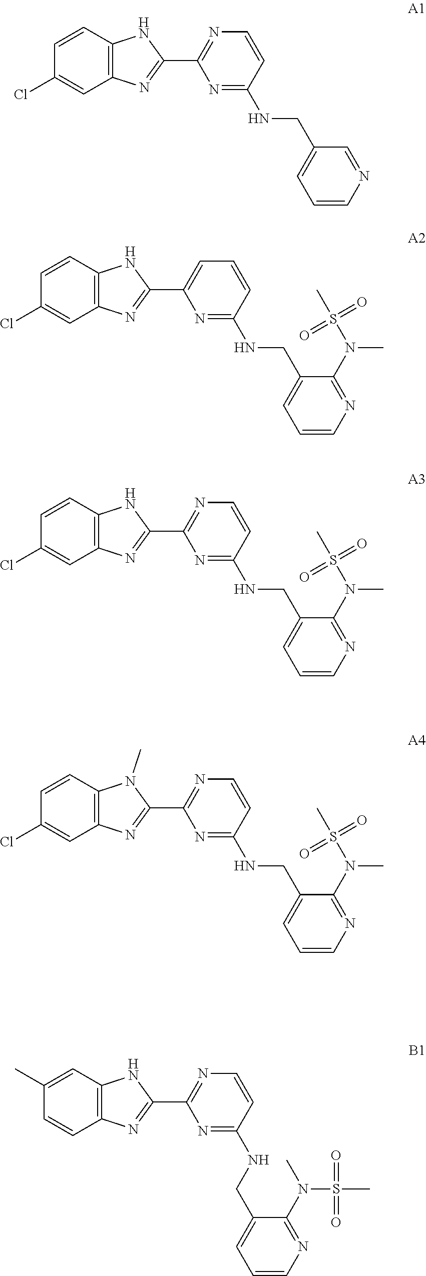 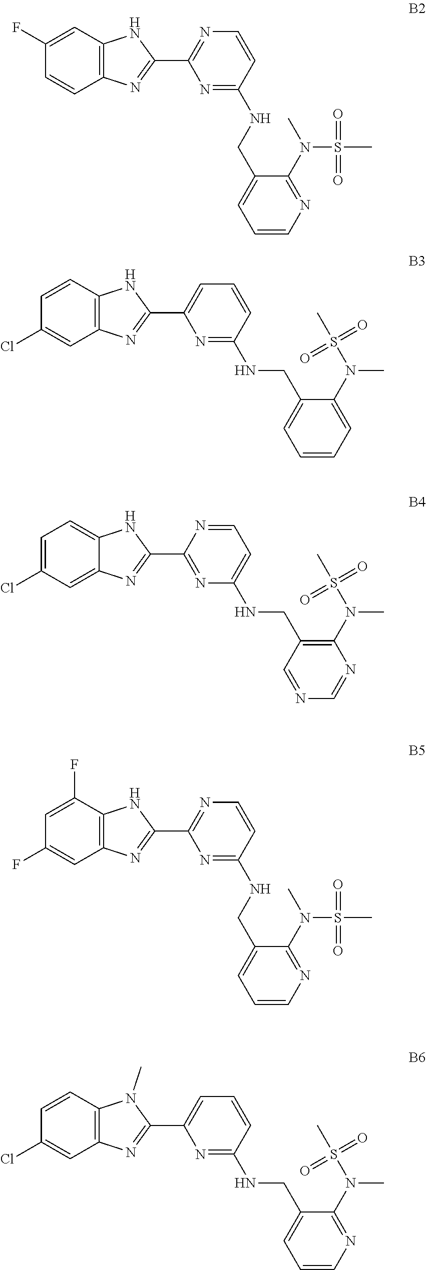 [SMILES: CN(c1ncccc1CNc1ccnc(-c2nc3ccc(F)cc3[nH]2)n1)S(C)(=O)=O ; CN(c1ccccc1CNc1cccc(-c2nc3cc(Cl)ccc3[nH]2)n1)S(C)(=O)=O ; CN(c1ncncc1CNc1ccnc(-c2nc3cc(Cl)ccc3[nH]2)n1)S(C)(=O)=O ; CN(c1ncccc1CNc1ccnc(-c2nc3cc(F)cc(F)c3[nH]2)n1)S(C)(=O)=O ; CN(c1ncccc1CNc1cccc(-c2nc3cc(Cl)ccc3n2C)n1)S(C)(=O)=O] 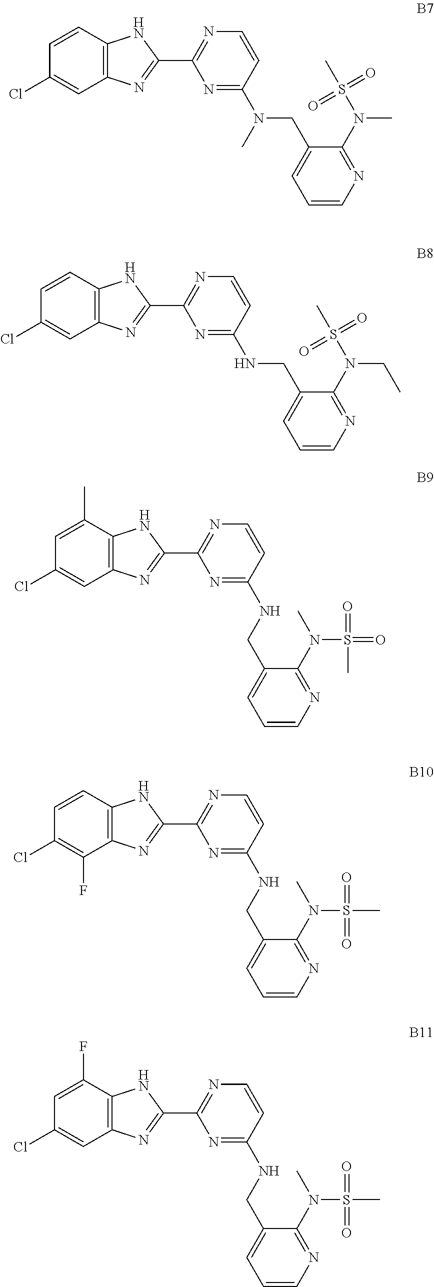 [SMILES: CN(Cc1cccnc1N(C)S(C)(=O)=O)c1ccnc(-c2nc3cc(Cl)ccc3[nH]2)n1 ; CCN(c1ncccc1CNc1ccnc(-c2nc3cc(Cl)ccc3[nH]2)n1)S(C)(=O)=O ; Cc1cc(Cl)cc2nc(-c3nccc(NCc4cccnc4N(C)S(C)(=O)=O)n3)[nH]c12 ; CN(c1ncccc1CNc1ccnc(-c2nc3c(F)c(Cl)ccc3[nH]2)n1)S(C)(=O)=O ; CN(c1ncccc1CNc1ccnc(-c2nc3cc(Cl)cc(F)c3[nH]2)n1)S(C)(=O)=O] 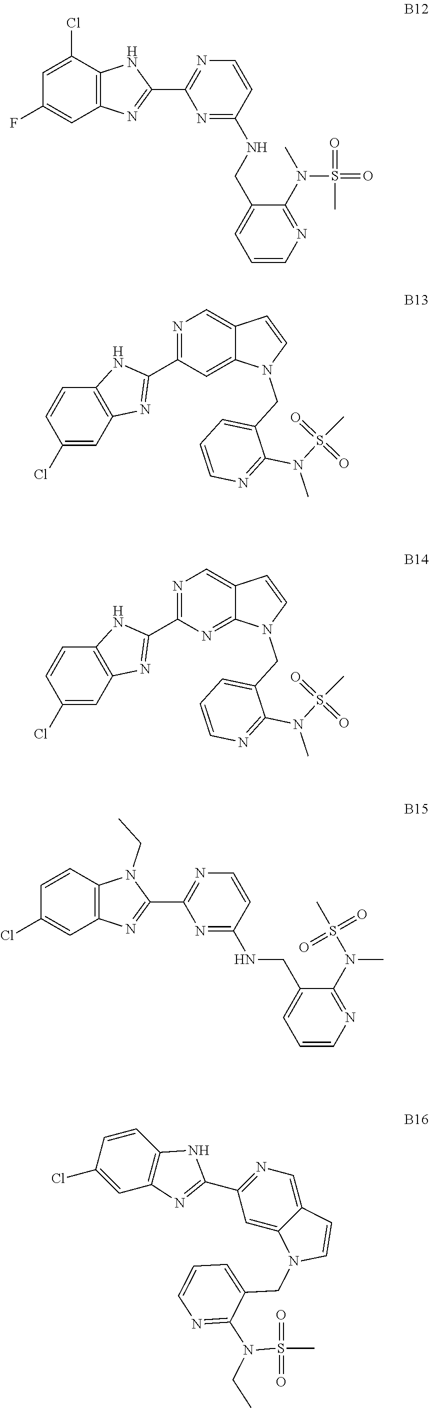 [SMILES: CN(c1ncccc1CNc1ccnc(-c2nc3cc(F)cc(Cl)c3[nH]2)n1)S(C)(=O)=O ; CN(c1ncccc1Cn1ccc2cnc(-c3nc4cc(Cl)ccc4[nH]3)cc21)S(C)(=O)=O ; CN(c1ncccc1Cn1ccc2cnc(-c3nc4cc(Cl)ccc4[nH]3)nc21)S(C)(=O)=O ; CCn1c(-c2nccc(NCc3cccnc3N(C)S(C)(=O)=O)n2)nc2cc(Cl)ccc21 ; CCN(c1ncccc1Cn1ccc2cnc(-c3nc4cc(Cl)ccc4[nH]3)cc21)S(C)(=O)=O] 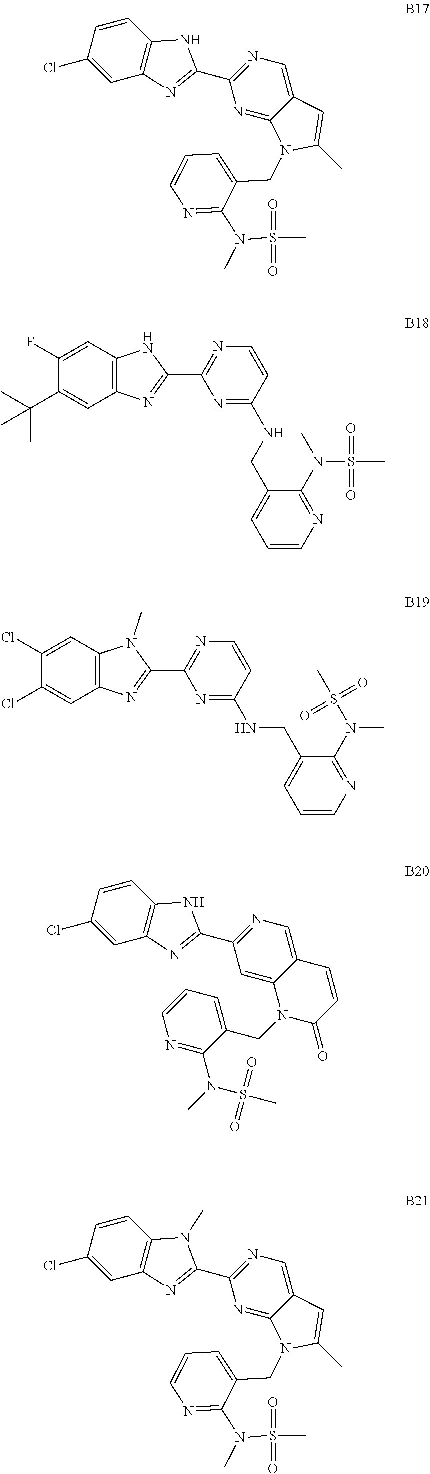 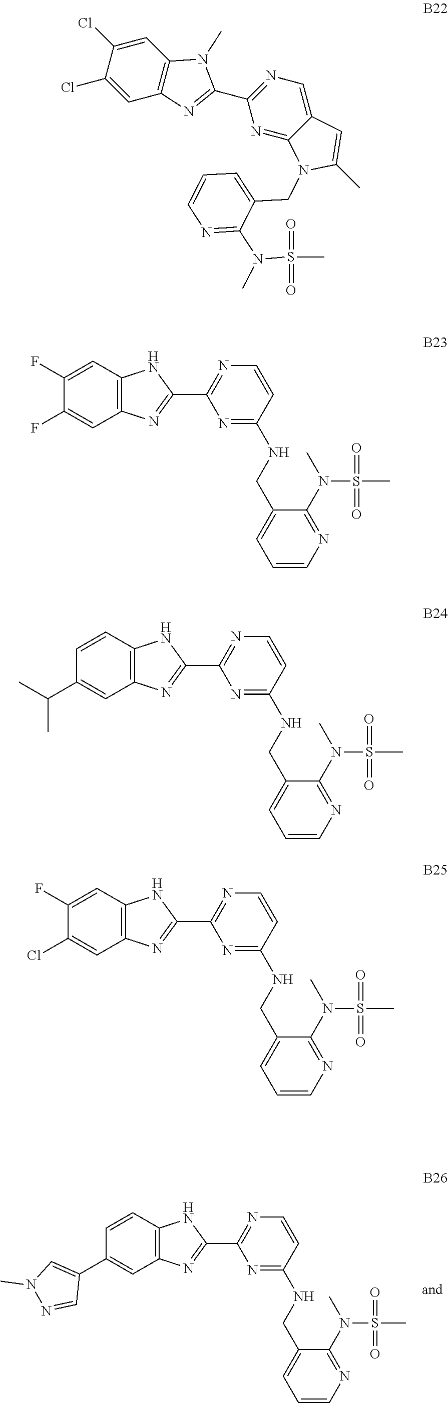 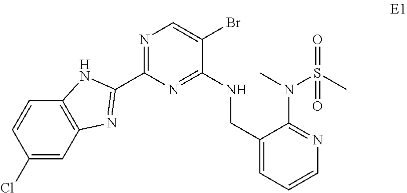 [SMILES: CN(c1ncccc1CNc1nc(-c2nc3cc(Cl)ccc3[nH]2)ncc1Br)S(C)(=O)=O]
and a physiologically acceptable salt thereof.

8. A method for manufacturing a compound of formula (T), comprising
(a) reacting a compound of formula (II) 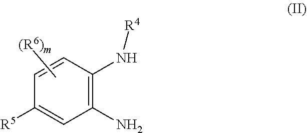
wherein R⁴, R⁵, R² and m have the meaning as defined in claim 1,
with a compound of formula (III) 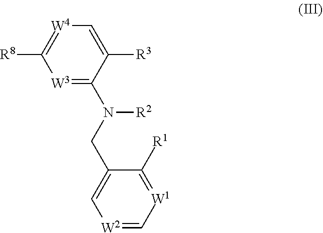
wherein
R⁸ denotes CN, COOH or Hal; and
W¹, W², W³, W⁴, R¹, R² and R³ have the meaning as defined in claim 1,
to yield a compound of formula (I) 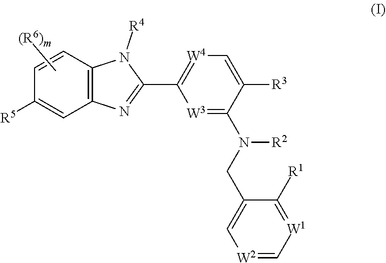
wherein W¹, W², W³, W⁴, R¹, R², R³, R⁴, R⁵, R⁶ and in have the meaning as defined in claim 1;
and optionally
(b) converting a base or an acid of the compound of formula (I) into a salt thereof.

9. A medicament, comprising,
at least one compound according to claim 1 and/or a physiologically acceptable salt thereof.

10. A pharmaceutical composition, comprising:
as active ingredient at least one compound according to claim 1 and/or a physiologically acceptable salt thereof together with pharmaceutically tolerable excipients, optionally in combination with one or more further active ingredients.

11. A method for the prophylactic or therapeutic treatment and/or monitoring of a disease that is caused, mediated and/or propagated by SRPK activity, the method comprising:
treating a subject in need thereof with the compound according to claim 1 and/or a physiologically acceptable salt thereof.

12. A method for the preparation of a medicament for the prophylactic or therapeutic treatment and/or monitoring of a disease that is caused, mediated and/or propagated by SRPK activity, comprising:
combining the compound according to claim 1 and/or a physiologically acceptable salt thereof with at least one solid, fluid and/or semi-fluid carrier or excipient and optionally in conjunction with a single or more other active substances in an appropriate dosage form.

13. A method for treating a disease that is caused, mediated and/or propagated by SRPK activity, wherein at least one compound according to claim 1 and/or a physiologically acceptable salt thereof is administered to a mammal in need of treatment.

14. The method according to claim 13, wherein the disease is selected from the group consisting of hyperproliferative disorders, cancer, metastases, tumors, angiogenesis disorders, tumor angiogenesis, benign hyperplasia, hemangioma, glioma, melanoma, Kaposi's sarcoma, prostate diseases related to vasculogenesis or angiogenesis, inflammation, pancreatitis, retinopathy, retinopathy of prematurity, diabetic retinopathy, diabetes, pain, restenosis, psoriasis, eczema, scleroderma and age-related macular degeneration.

15. A method for inhibiting SRPK, wherein a system expressing SRPK is contacted with at least one compound according to claim 1 and/or a physiologically acceptable salt thereof under conditions such that the SRPK is inhibited.
